SEQUENCE LISTING (53 sequences)

SEQ ID NO: 1 - DNA, 13 bp, Artificial Sequence
  (sequence is synthesized) primer sequence
gagaggcctgagt

SEQ ID NO: 2 - DNA, 16 bp, Artificial Sequence
  (sequence is synthesized) primer sequence
aggagaggcctgagta

SEQ ID NO: 3 - DNA, 18 bp, Artificial Sequence
  (sequence is synthesized) primer sequence
caggagaggcctgagtat

SEQ ID NO: 4 - DNA, 20 bp, Artificial Sequence
  (sequence is synthesized) primer sequence
gcaggagaggcctgagtatt

SEQ ID NO: 5 - DNA, 22 bp, Artificial Sequence
  (sequence is synthesized) primer sequence
agcaggagaggcctgagtattg

SEQ ID NO: 6 - DNA, 24 bp, Artificial Sequence
  (sequence is synthesized) primer sequence
gagcaggagaggcctgagtattgg

SEQ ID NO: 7 - DNA, 26 bp, Artificial Sequence
  (sequence is synthesized) primer sequence
agagcaggagaggcctgagtattggg

SEQ ID NO: 8 - DNA, 28 bp, Artificial Sequence
  (sequence is synthesized) primer sequence
tagagcaggagaggcctgagtattggga

SEQ ID NO: 9 - DNA, 30 bp, Artificial Sequence
  (sequence is synthesized) primer sequence
atagagcaggagaggcctgagtattgggac

SEQ ID NO: 10 - DNA, 32 bp, Artificial Sequence
  (sequence is synthesized) primer sequence
gatagagcaggagaggcctgagtattgggacc

SEQ ID NO: 11 - DNA, 19 bp, Artificial Sequence
  (sequence is synthesized) primer sequence
actcggtcaatctgtgagt

SEQ ID NO: 12 - DNA, 21 bp, Artificial Sequence
  (sequence is synthesized) primer sequence
cactcggtcaatctgtgagtg

SEQ ID NO: 13 - DNA, 23 bp, Artificial Sequence
  (sequence is synthesized) primer sequence
ccactcggtcaatctgtgagtgg

SEQ ID NO: 14 - DNA, 25 bp, Artificial Sequence
  (sequence is synthesized) primer sequence
tccactcggtcaatctgtgagtggg

SEQ ID NO: 15 - DNA, 27 bp, Artificial Sequence
  (sequence is synthesized) primer sequence
gtccactcggtcaatctgtgagtgggc

SEQ ID NO: 16 - DNA, 29 bp, Artificial Sequence
  (sequence is synthesized) primer sequence
ggtccactcggtcaatctgtgagtgggcc

SEQ ID NO: 17 - DNA, 31 bp, Artificial Sequence
  (sequence is synthesized) primer sequence
aggtccactcggtcaatctgtgagtgggcct

SEQ ID NO: 18 - DNA, 33 bp, Artificial Sequence
  (sequence is synthesized) primer sequence
caggtccactcggtcaatctgtgagtgggcctt

SEQ ID NO: 19 - DNA, 35 bp, Artificial Sequence
  (sequence is synthesized) primer sequence
ccaggtccactcggtcaatctgtgagtgggccttc

SEQ ID NO: 20 - DNA, 13 bp, Artificial Sequence
  (sequence is synthesized) primer sequence
agacacggaatgt

SEQ ID NO: 21 - DNA, 13 bp, Artificial Sequence
  (sequence is synthesized) primer sequence
acattccgtgtct

SEQ ID NO: 22 - DNA, 15 bp, Artificial Sequence
  (sequence is synthesized) primer sequence
gagacacggaatgtg

SEQ ID NO: 23 - DNA, 15 bp, Artificial Sequence
  (sequence is synthesized) primer sequence
cacattccgtgtctc

SEQ ID NO: 24 - DNA, 17 bp, Artificial Sequence
  (sequence is synthesized) primer sequence
ggagacacggaatgtga

SEQ ID NO: 25 - DNA, 17 bp, Artificial Sequence
  (sequence is synthesized) primer sequence
tcacattccgtgtctcc

SEQ ID NO: 26 - DNA, 19 bp, Artificial Sequence
  (sequence is synthesized) primer sequence
aggagacacggaatgtgaa

SEQ ID NO: 27 - DNA, 19 bp, Artificial Sequence
  (sequence is synthesized) primer sequence
ttcacattccgtgtctcct

SEQ ID NO: 28 - DNA, 21 bp, Artificial Sequence
  (sequence is synthesized) primer sequence
caggagacacggaatgtgaag

SEQ ID NO: 29 - DNA, 21 bp, Artificial Sequence
  (sequence is synthesized) primer sequence
cttcacattccgtgtctcctg

SEQ ID NO: 30 - DNA, 23 bp, Artificial Sequence
  (sequence is synthesized) primer sequence
ccaggagacacggaatgtgaagg

SEQ ID NO: 31 - DNA, 23 bp, Artificial Sequence
  (sequence is synthesized) primer sequence
ccttcacattccgtgtctcctgg

SEQ ID NO: 32 - DNA, 26 bp, Artificial Sequence
  (sequence is synthesized) primer sequence
gaccaggagacacggaatgtgaaggc

SEQ ID NO: 33 - DNA, 26 bp, Artificial Sequence
  (sequence is synthesized) primer sequence
gccttcacattccgtgtctcctggtc

SEQ ID NO: 34 - DNA, 28 bp, Artificial Sequence
  (sequence is synthesized) primer sequence
ggaccaggagacacggaatgtgaaggcc

SEQ ID NO: 35 - DNA, 28 bp, Artificial Sequence
  (sequence is synthesized) primer sequence
ggccttcacattccgtgtctcctggtcc

SEQ ID NO: 36 - DNA, 30 bp, Artificial Sequence
  (sequence is synthesized) primer sequence
gggaccaggagacacggaatgtgaaggccc

SEQ ID NO: 37 - DNA, 30 bp, Artificial Sequence
  (sequence is synthesized) primer sequence
gggccttcacattccgtgtctcctggtccc

SEQ ID NO: 38 - DNA, 19 bp, Artificial Sequence
  (sequence is synthesized) primer sequence
gatagagcaggagaggcct

SEQ ID NO: 39 - DNA, 18 bp, Artificial Sequence
  (sequence is synthesized) primer sequence
ccaggtccactcggtcaa

SEQ ID NO: 40 - DNA, 16 bp, Artificial Sequence
  (sequence is synthesized) detection reagent
agacacggaatgtgaa

SEQ ID NO: 41 - DNA, 16 bp, Artificial Sequence
  (sequence is synthesized) detection reagent
ttcacattccgtgtct

SEQ ID NO: 42 - DNA, 17 bp, Artificial Sequence
  (sequence is synthesized) primer sequence
gaggccacagctgcaac

SEQ ID NO: 43 - DNA, 18 bp, Artificial Sequence
  (sequence is synthesized) primer sequence
gttggccacgttgtccat

SEQ ID NO: 44 - DNA, 18 bp, Artificial Sequence
  (sequence is synthesized) primer sequence
atacgagtcattcaattc

SEQ ID NO: 45 - DNA, 18 bp, Artificial Sequence
  (sequence is synthesized) primer sequence
gaattgaatgactcgtat

SEQ ID NO: 46 - DNA, 21 bp, Artificial Sequence
  (sequence is synthesized) primer sequence
ctgagaggcaagagttgttcc

SEQ ID NO: 47 - DNA, 19 bp, Artificial Sequence
  (sequence is synthesized) primer sequence
gcaggtgcctttgcagaaa

SEQ ID NO: 48 - DNA, 51 bp, Artificial Sequence
  (sequence is synthesized) primer sequence
  modified base 4: a, t, c or g
ctgnccttccctttgtgacttgaagaaccctgactttgtttctgcaaaggc

SEQ ID NO: 49 - DNA, 51 bp, Artificial Sequence
  (sequence is synthesized) primer sequence
  modified base 48: a, t, c or g
gcctttgcagaaacaaagtcagggtacttcaagtcacaaagggaaggncag

SEQ ID NO: 50 - DNA, 70 bp, Artificial Sequence
  (sequence is synthesized) primer sequence
cgcgggcgccgtggatagagcaggagaggcctgagtattgggaccaggagacacggaatgtgaaggccca

SEQ ID NO: 51 - DNA, 71 bp, Artificial Sequence
  (sequence is synthesized) primer sequence
ctcacagattgaccgagtggacctggggaccctgcgcggctactacaaccagagcgaggccggtgagtgac

SEQ ID NO: 52 - DNA, 51 bp, Artificial Sequence
  (sequence is synthesized) primer sequence
  modified base 4: a, t, c or g
ctgnccttccctttgtgacttgaagtaccctgactttgtttctgcaaaggc

SEQ ID NO: 53 - DNA, 51 bp, Artificial Sequence
  (sequence is synthesized) primer sequence
  modified base 48: a, t, c or g
gcctttgcagaaacaaagtcagggttcttcaagtcacaaagggaaggncag

(19) United States
(12) Patent Application Publication
(10) Pub. No.: US 2022/0017948 A1
(43) Pub. Date: Jan. 20, 2022

(54) DETECTION OF SPECIFIC HLA ALLELES

(71) Applicant: GENOMIND, INC., King of Prussia, PA (US)

(72) Inventors: Kathleen DAVIS, King of Prussia, PA (US); David ROBBINS, King of Prussia, PA (US)

(21) Appl. No.: 17/353,234

(22) Filed: Jun. 21, 2021

Related U.S. Application Data
(60) Provisional application No. 63/042,902, filed on Jun. 23, 2020.

(51) Int. Cl. C12Q 1/6818; C12Q 1/686; C12N 9/22; G01N 33/58; G01N 21/64

(52) U.S. Cl. CPC C12Q 1/6818; C12Q 1/686; G01N 2021/6439; G01N 33/582; G01N 21/6428; C12N 9/22

(57) ABSTRACT

A screening method is provided for a specific HLA allele in an individual, including but not limited to an HLA-A*31:01 allele. Also included are methods for treating an individual in need of a medication, which can induce ADRs associated with a specific HLA allele, and a kit including detection reagents used in the screening method.

### I. CROSS-REFERENCE

This application claims priority to U.S. Provisional Application No. 63/042,902 filed Jun. 23, 2020, the disclosure of which is hereby incorporated by reference, in its entirety, for all purposes.

### II. SEQUENCE LISTING

The instant application contains a Sequence Listing which has been submitted electronically in ASCII format and is hereby incorporated by reference in its entirety. Said ASCII copy, created on Jun. 21, 2021, is named 122658_5009_sequence_listing.txt and is 13 kilobytes in size.

### III. BACKGROUND OF THE INVENTION

Adverse drug reactions (ADRs) remain a common and major problem in healthcare. Severe ADRs may result in withdrawal of a drug from the market, despite clinical utility of the drug for the majority of patients. Recent studies demonstrate that ADRs possess strong genetic predisposition. Most prominently, class I and class II human leucocyte antigen (HLA) alleles have been linked with ADRs in various ethnic populations. For example, HLA-A*31:01 is associated with ADRs from carbamazepine (McCormack M, et al., N Engl J Med. 2011; 364:1134-43; Ozeki T et al., Hum Mol Genet. 2011; 20:1034-41); HLA-A*33:03, HLA-B*58:01 or HLA-C*03:02 is associated with ADRs from allopurinol (Cristallo A F, Int J Immunogenet. 2011; 38:303-9; Kang H R, Pharmacogenet Genomics. 2011; 21:303-7.); HLA-B*13:01 is associated with ADRs from dapsone (Zhang FR, N Engl J Med. 2013; 369:1620-8.); HLA-B*15:02 is associated with ADRs from carbamazepine and phenytoin (Chung W H, Nature. 2004; 428:486.); HLA-B*15:11 is associated with ADRs from carbamazepine (Kaniwa N, Epilepsia. 2010; 51:2461-5); HLA-B*35:01 is associated with ADRs from nevirapine (Cornejo Castro E M, Pharmacogenet Genomics. 2015; 25:186-98.); HLA-B*38:02 is associated with ADRs from carbimazole and methimazole (Chen P L, Nat Commun. 2015; 6:7633); HLA-B*40:01 is associated with ADRs from carbamazepine (Hung S I, Pharmacogenet Genomics. 2006; 16:297-30); HLA-B*57:01 is associated with ADRs from abacavir (Mallal S, N Engl J Med. 2008; 358:568-79.); HLA-B*59:01 and HLA-C*01:02 are associated with ADRs from methazolamide (Kim S H, Pharmacogenomics. 2010; 11:879-84.); HLA-DRB1*01:01 is associated with ADRs from nevirapine (Cornejo Castro E M, Pharmacogenet Genomics. 2015; 25:186-98); and HLA-DRB1*07:01 is associated with ADRs from lapatinib (Schaid D J, J Clin Oncol. 2014; 32:2296-303).

Methods for determining a specific HLA allele include conventional genotyping methods, for example next-generation sequencing. However, since genome sequencing is labor intensive and expensive, it is mostly commonly used for organ transplant or other medically necessary situations.

Methods for screening a specific HLA allele for determining ADRs can include detecting single nucleotide polymorphisms (SNPs), which is based on strong linkage disequilibrium between a HLA variant and one or more SNPs. However, most reported SNPs have not been validated, and there is concern regarding clinical utility of this approach due to error rate and inconsistency across different ethnic populations.

This invention addresses the existing issues with screening of a specific HLA allele, and provides a more specific and cost effective method for screening a specific HLA allele for predicting ADRs.

### IV. BRIEF SUMMARY OF THE INVENTION

In one aspect, the present invention discloses a method of determining whether an individual carries an HLA-A*31:01 allele. The method comprise:

(A) performing a first polymerase chain reaction (PCR) assay designed to amplify a first genomic region from a biological sample obtained from the individual, the first genomic region encompassing the loci corresponding to SNPs rs1059449, rs41541222, rs1059457, rs79361534, and rs1059471, using a first set of PCR primers that flank the first genomic region, wherein the first set of PCR primers comprises at least one of (i) a first primer that overlaps the locus corresponding to rs1059449 and is specific for the rs1059449 G>A SNP or (ii) a second primer that overlaps the locus corresponding to rs1059471 and is specific for the rs1059471 C>T SNP; (B) contacting the first PCR assay with the set of one or more HLA-A*31:01 detection reagents, wherein each respective detection reagent in the set of one or more HLA-A*31:01 detection reagents comprises a moiety that is detectable when the first genomic region is amplified and the respective detection reagent is capable of specifically binding to the first genomic region; and (C) attempting to detect the set of one or more HLA-A*31:01 detection reagents, wherein a first detection reagent in the set of one or more HLA-A*31:01 detection reagents is specific for the presence of the reference G allele at the locus corresponding to the rs1059457 SNP, and a second detection reagent in the set of one or more HLA-A*31:01 detection reagents is specific for the presence of the reference G allele at the locus corresponding to the rs79361534 SNP, when the entire set of one or more HLA-A*31:01 detection reagents is detected, the individual is deemed to be a carrier of the HLA-A*31:01 allele, and when less than the entire set of one or more HLA-A*31:01 detection reagents is detected, the individual is deemed not to be a carrier of the HLA-A*31:01 allele, thereby determining whether the individual carries the HLA-A*31:01 allele. In some embodiments, when the first set of PCR primers includes the first primer, the locus corresponding to rs41541222 is either (i) overlapped by the first primer, wherein the first primer is specific for the rs41541222 G>T SNP or (ii) overlapped by a respective detection reagent in a set of one or more HLA-A*31:01 detection reagents, wherein the respective detection reagent overlaps with the locus corresponding to rs41541222 and is specific for the rs41541222 G>T SNP; and the locus corresponding to rs1059471 is either (i) overlapped by the second allele-specific primer, which is included in the first set of primers or (ii) overlapped by a respective detection reagent in the set of one or more HLA-A*31:01 detection reagents, wherein the respective detection reagent overlaps with rs1059471 and is specific for the rs1059471 C>T SNP. In some embodiments, when the first set of PCR primers includes the second primer, the locus corresponding to rs1059449 is either (i) overlapped by the first primer, which is included in the first set of primers or (ii) overlapped by a respective detection reagent in the set of one or more HLA-A*31:01 detection reagents, wherein the respective detection reagent overlaps with rs1059449 and is specific for the rs1059449 G>A SNP; and the locus corresponding to rs41541222 is either (i) overlapped by the first primer, wherein the first primer is specific for the rs41541222 G>T SNP or (ii) overlapped by a respective detection reagent in the set of one or more HLA-A*31:01 detection reagents, wherein the respective detection reagent overlaps with rs41541222 and is specific for the rs41541222 G>T SNP.

In some embodiments, the first genomic region does not encompass the locus corresponding to the rs41562315 SNP.

In some embodiments, the first primer overlaps with the locus corresponding to rs41541222 and is specific for the rs41541222 G>T SNP.

In some embodiments, the first primer has a nucleotide sequence comprising at least 10 consecutive nucleotides of SEQ ID NO:1. In some embodiments, the first primer comprises a nucleic acid sequence of SEQ ID NO:38

In some embodiments, the second primer has a nucleotide sequence comprising at least 10 consecutive nucleotides of SEQ ID NO:11. In some embodiments, the second primer comprises a nucleic acid sequence of SEQ ID NO:39.

In some embodiments, the first set of PCR primers consists of the first primer and the second primer.

In some embodiments, the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP. For example, the single HLA-A*31:01 detection reagent can comprises a nucleic acid sequence of SEQ ID NO:40 or SEQ ID NO:41.

In some embodiments, each respective detection reagent in the set of one or more HLA-A*31:01 detection reagents is conjugated to one or more detection label. In some embodiments, the one or more detection label comprises a fluorescent dye. In some embodiments, the one or more detection label comprises a matching pair of electronic energy transfer fluorophores comprising a donor fluorophore and a quencher fluorophore for the donor fluorophore.

In some embodiments, step (B) of the method comprises adding the set of one or more HLA-A*31:01 detection reagents to the PCR assay prior to performing step (A). In some embodiments, step (B) of the method comprises contacting the set of one or more HLA-A*31:01 detection reagents with a product of the PCR assay performed in step (A).

In some embodiments, the first PCR assay is a quantitative real-time PCR assay. In some embodiments, the first PCR assay is a multiplex PCR assay using a second set of PCR primers designed to amplify a control genomic locus from the biological sample obtained from the individual; and the first PCR assay is contacted with a control detection reagent that binds to the control genomic locus. In some embodiments, the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP, wherein the single HLA-A*31:01 detection reagent is conjugated to a first matching pair of electronic energy transfer fluorophores comprising a first donor fluorophore and a first quencher fluorophore for the first donor fluorophore; and the control detection reagent is conjugated to a second matching pair of electronic energy transfer fluorophores comprising a second donor fluorophore and a second quencher fluorophore for the second donor fluorophore. In some embodiments, the first and the second matching pairs of electronic energy transfer fluorophores are selected from the group consisting of FAM-TAMRA, VIC-TAMRA, TET-TAMRA and JOE-TAMRA, and wherein the first and the second matching pairs of electronic energy transfer fluorophores are different.

In some embodiments, the method of determining whether an individual carries an HLA-A*31:01 allele described above further comprises a step of determining whether the individual is a carrier of the rs1061235 A>T SNP. When it is determined that the individual does not carry the rs1061235 A>T SNP, the individual is deemed not to be a carrier of the HLA-A*31:01 allele.

In another aspect, the present invention discloses a method of predicting whether an individual will have an adverse drug reaction to carbamazepine, according to the method for determining whether the individual carries an HLA-A*31:01 allele described herein. In some embodiments, the individual has a condition selected from the group consisting of epilepsy, trigeminal neuralgia, acute manic and mixed episodes in bipolar I disorder, neuropathic pains, fibromyalgia, and refractory schizophrenia.

In another aspect, the present invention provides a method of treating an individual in need of carbamazepine. The method comprises determining whether the individual carries an HLA-A*31:01 allele by: (A) screening the genomic loci corresponding to the SNPs rs1059449, rs41541222, rs1059457, rs79361534, and rs1059471, and (B) determining the individual carries all five of a rs1059449 G>A SNP, a rs41541222 G>T SNP, a reference G allele at the locus corresponding to the rs1059457 SNP, a reference G allele at the locus corresponding to the rs79361534 SNP, and a rs1059471 C>T SNP, wherein the presence of all five indicates that the individual carries an HLA-A*31:01 allele. If the individual does not carry an HLA-A*31:01 allele, then administering carbamazepine to the individual, and if the individual does carry an HLA-A*31:01 allele, then administering an alternative medication to carbamazepine to the individual.

In another aspect, the present invention provides a method of providing clinical support for the treatment of an individual in need of carbamazepine, comprising determining whether the individual carries an HLA-A*31:01 allele by: (A) screening the genomic loci corresponding to the SNPs rs1059449, rs41541222, rs1059457, rs79361534, and rs1059471, and (B) determining the individual carries all five of a rs1059449 G>A SNP, a rs41541222 G>T SNP, a reference G allele at the locus corresponding to the rs1059457 SNP, a reference G allele at the locus corresponding to the rs79361534 SNP, and a rs1059471 C>T SNP, wherein the presence of all five indicates that the individual carries an HLA-A*31:01 allele; and transmitting, to a health care professional, a report recommending one or more possible treatment regimens for the individual, wherein when the individual does not carry an HLA-A*31:01 allele, then the one or more possible treatment regimens recommended in the report includes administration of carbamazepine to the individual, and wherein when the individual does carry an HLA-A*31:01 allele, then the one or more possible treatment regimens recommended in the report does not include administration of carbamazepine to the individual.

In some embodiments, the method of treating an individual in need of carbamazepine and the method of providing clinical support for the treatment of an individual in need of carbamazepine described herein comprises (A) performing a first polymerase chain reaction (PCR) assay designed to amplify a first genomic region from a biological sample obtained from the individual, the first genomic region encompassing the loci corresponding to SNPs rs1059449, rs41541222, rs1059457, rs79361534, and rs1059471, using a first set of PCR primers that flank the first genomic region, wherein the first set of PCR primers comprises at least one of (i) a first primer that overlaps the locus corresponding to rs1059449 and is specific for the rs1059449 G>A SNP or (ii) a second primer that overlaps the locus corresponding to rs1059471 and is specific for the rs1059471 C>T SNP; (B) contacting the first PCR assay with the set of one or more HLA-A*31:01 detection reagents, wherein each respective detection reagent in the set of one or more HLA-A*31:01 detection reagents comprises a moiety that is detectable when the first genomic region is amplified and the respective detection reagent is capable of specifically binding to the first genomic region; and (C) attempting to detect the set of one or more HLA-A*31:01 detection reagents, wherein a first detection reagent in the set of one or more HLA-A*31:01 detection reagents is specific for the presence of the reference G allele at the locus corresponding to the rs1059457 SNP, and a second detection reagent in the set of one or more HLA-A*31:01 detection reagents is specific for the presence of the reference G allele at the locus corresponding to the rs79361534 SNP. When the entire set of one or more HLA-A*31:01 detection reagents is detected, the individual is deemed to be a carrier of the HLA-A*31:01 allele, and when less than the entire set of one or more HLA-A*31:01 detection reagents is detected, the individual is deemed not to be a carrier of the HLA-A*31:01 allele, thereby determining whether the individual carries the HLA-A*31:01 allele. In some embodiments, when the first set of PCR primers includes the first primer, the locus corresponding to rs41541222 is either (i) overlapped by the first primer, wherein the first primer is specific for the rs41541222 G>T SNP or (ii) overlapped by a respective detection reagent in a set of one or more HLA-A*31:01 detection reagents, wherein the respective detection reagent overlaps with the locus corresponding to rs41541222 and is specific for the rs41541222 G>T SNP, and the locus corresponding to rs1059471 is either (i) overlapped by the second allele-specific primer, which is included in the first set of primers or (ii) overlapped by a respective detection reagent in the set of one or more HLA-A*31:01 detection reagents, wherein the respective detection reagent overlaps with rs1059471 and is specific for the rs1059471 C>T SNP. In some embodiments, when the first set of PCR primers includes the second primer, the locus corresponding to rs1059449 is either (i) overlapped by the first primer, which is included in the first set of primers or (ii) overlapped by a respective detection reagent in the set of one or more HLA-A*31:01 detection reagents, wherein the respective detection reagent overlaps with rs1059449 and is specific for the rs1059449 G>A SNP, and the locus corresponding to rs41541222 is either (i) overlapped by the first primer, wherein the first primer is specific for the rs41541222 G>T SNP or (ii) overlapped by a respective detection reagent in the set of one or more HLA-A*31:01 detection reagents, wherein the respective detection reagent overlaps with rs41541222 and is specific for the rs41541222 G>T SNP.

In some embodiments of the methods described above, the first genomic region does not encompass the locus corresponding to the rs41562315 SNP.

In some embodiments of the methods described above, the first set of PCR primers comprises a first allele-specific primer that (i) overlaps the locus corresponding to rs1059449 and is specific for the rs1059449 G>A SNP and (ii) overlaps the locus corresponding to rs41541222 and is specific for the rs41541222 G>T SNP.

In some embodiments of the methods described above, the first set of PCR primers comprises a first allele-specific primer having a nucleotide sequence comprising at least 10 consecutive nucleotides of SEQ ID NO:1. In some embodiments, the first allele-specific primer comprises a nucleic acid sequence of SEQ ID NO:38.

In some embodiments of the methods described above, the first set of allele-specific PCR primers consists of the first allele-specific primer and the second allele-specific primer.

In some embodiments of the methods described above, the first set of PCR primers comprises a second allele-specific primer that has a nucleotide sequence comprising at least 10 consecutive nucleotides of SEQ ID NO:11. In some embodiments, the second allele-specific primer comprises a nucleic acid sequence of SEQ ID NO:39.

In some embodiments of the methods described above, the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP. In some embodiments, the single HLA-A*31:01 detection reagent comprises a nucleic acid sequence of SEQ ID NO:40 or SEQ ID NO:41.

In some embodiments of the methods described above, each respective detection reagent in the set of one or more HLA-A*31:01 detection reagents is conjugated to one or more detection label. In some embodiments, the one or more detection label comprises a fluorescent dye. In some embodiments, the one or more detection label comprises a matching pair of electronic energy transfer fluorophores comprising a donor fluorophore and a quencher fluorophore for the donor fluorophore.

In some embodiments of the methods described above, contacting in step (B) comprises adding the set of one or more HLA-A*31:01 detection reagents to the PCR assay prior to the performing step (A).

In some embodiments of the methods described above, contacting in step (B) comprises contacting the set of one or more HLA-A*31:01 detection reagents with a product of the PCR assay performed in step (A).

In some embodiments of the methods described above, the first PCR assay is a quantitative real-time PCR assay. In some embodiments, the first PCR assay is a multiplex PCR assay using a second set of PCR primers designed to amplify a control genomic locus from the biological sample obtained from the individual; and the first PCR assay is contacted with a control detection reagent that binds to the control genomic locus.

In some embodiments of the methods described above, the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP; the single HLA-A*31:01 detection reagent is conjugated to a first matching pair of fluorophores comprising a first donor fluorophore and a first quencher fluorophore for the first donor fluorophore; and the control detection reagent is conjugated to a second matching pair of electronic energy transfer fluorophores comprising a second donor fluorophore and a second quencher fluorophore for the second donor fluorophore. In some embodiments, the first and the second matching pairs of electronic energy transfer fluorophores are selected from the group consisting of FAM-TAMRA, VIC-TAMRA, TET-TAMRA and JOE-TAMRA, and the first and the second matching pairs of electronic energy transfer fluorophores are different.

In some embodiments, the method of treating an individual in need of carbamazepine and the method of providing clinical support for the treatment of an individual in need of carbamazepine described herein further comprises determining whether the individual is a carrier of an rs1061235 A>T SNP. In some embodiments, the individual has a condition selected from the group consisting of epilepsy, trigeminal neuralgia, acute manic and mixed episodes in bipolar I disorder, neuropathic pains, fibromyalgia, and refractory schizophrenia.

In another aspect, the current invention provides a method of determining whether an individual carries a specific allele of an HLA gene, comprising (A) performing a real time polymerase chain reaction (PCR) assay designed to amplify a first genomic region of the HLA gene from a biological sample obtained from the individual, wherein the first genomic region comprises a set of two or more loci that distinguishes the specific allele of the HLA gene from other alleles of the HLA gene, wherein the PCR assay comprises a first set of PCR primers that flank the first genomic region and a set of nucleic acid probes that is capable of binding specifically to the first genomic region, wherein the first set of PCR primers comprises a first allele-specific primer that overlaps with at least one locus in the set of two or more loci and is specific for the nucleotide at the locus corresponding to the specific allele of the HLA gene, wherein set of nucleic acid probes overlaps with at least one locus in the set of two or more loci not overlapped by the first set of PCR primers and is specific for the nucleotide at the locus corresponding to the specific allele of the HLA gene, and each probe from the set of nucleic acid probes is conjugated with one or matching pair of electronic energy transfer fluorophores comprising an excitation fluorophore and a quenching fluorophore for the excitation fluorophore, and wherein the PCR primers and the set of nucleic acid probes in combination overlap with all of the set of two or more loci and are specific for the nucleotides at the loci corresponding to the specific allele of the HLA gene; and (B) attempting to detect the set of one or more nucleic acid probes. When the entire set of one or more nucleic acid probes is detected, the individual is deemed to be a carrier of the allele of the HLA gene, and when less than the entire set of one or more nucleic acid probes is detected, the individual is deemed not to be a carrier of the allele of the HLA gene, thereby determining whether the individual carries the allele of the HLA gene.

In some embodiments of the method, the set of two or more loci comprises a set of three or more loci. In some embodiments, the first set of PCR primers comprises a second allele-specific primer that overlaps at least one locus in the set of three or more loci and is specific for the nucleotide at the locus corresponding to the specific allele of the HLA gene.

In some embodiments of the method, the PCR assay is a multiplex PCR assay using a second set of PCR primers designed to amplify a control genomic locus from the biological sample obtained from the individual; and the PCR reaction is contacted with a control nucleic acid probe that binds to the control locus. In some embodiments, the set of one or more nucleic acid probes consists of a single nucleic acid probe, wherein the single nucleic acid probe is conjugated to a first pair of electronic energy transfer fluorophores comprising a first donor fluorophore and a first quencher fluorophore for the first donor fluorophore, and the control detection reagent is conjugated to a second pair of electronic energy transfer fluorophores comprising a second donor fluorophore and a second quencher fluorophore for the second donor fluorophore. In some embodiments, the first and the second matching pairs of electronic energy transfer fluorophores are selected from the group consisting of FAM-TAMRA, VIC-TAMRA, TET-TAMRA and JOE-TAMRA, and the first and the second matching pairs of electronic energy transfer fluorophores are different.

In some embodiments, the method of determining whether an individual carries a specific allele of an HLA gene, further comprises determining whether the individual is a carrier of one or more additional SNPs that distinguishes the specific allele of the HLA gene from other alleles of the HLA gene, wherein the loci corresponding to the one or more additional SNPs are in a second genomic region that is not flanked by the first set of PCR primers.

In another aspect, the current invention provides a kit for determining whether an individual carries an HLA-A*31:01 allele. The kit comprises a first set of PCR primers designed to amplify a first genomic region from a biological sample obtained from the individual and a set of one or more HLA-A*31:01 detection reagents, wherein the first genomic region encompasses the loci corresponding to SNPs rs1059449, rs41541222, rs1059457, rs79361534, and rs1059471, wherein the first set of PCR primers comprises at least one of (i) a first allele-specific primer that overlaps the locus corresponding to rs1059449 and is specific for the rs1059449 G>A SNP, or (ii) a second allele-specific primer that overlaps the locus corresponding to rs1059471 and is specific for the rs1059471 C>T SNP. In some embodiments, when the first set of PCR primers includes the first allele-specific primer, the locus corresponding to rs41541222 is either (i) overlapped by the first allele-specific primer, wherein the first primer is specific for the rs41541222 G>T SNP, or (ii) overlapped by a respective detection reagent in a set of one or more HLA-A*31:01 detection reagents, wherein the respective detection reagent overlapping the locus corresponding to rs41541222 is specific for the presence of the rs41541222 G>T SNP; and the locus corresponding to rs1059471 is either (i) overlapped by the second allele-specific primer, which is included in the first set of primers, or (ii) overlapped by a respective detection reagent in the set of one or more HLA-A*31:01 detection reagents, wherein the respective detection reagent overlapping rs1059471 is specific for the rs1059471 C>T SNP. In some embodiments, when the first set of PCR primers includes the second allele-specific primer, the locus corresponding to rs1059449 is either (i) overlapped by the first allele-specific primer, which is included in the first set of primers, or (ii) overlapped by a respective detection reagent in the set of one or more HLA-A*31:01 detection reagents, wherein the respective detection reagent overlapping rs1059449 is specific for the rs1059449 G>A SNP; and the locus corresponding to rs41541222 is either (i) overlapped by the first allele-specific primer, wherein the first primer is specific for the rs41541222 G>T SNP, or (ii) overlapped by a respective detection reagent in the set of one or more HLA-A*31:01 detection reagents, wherein the respective detection reagent overlapping rs41541222 is specific for the presence of the rs41541222 G>T SNP. In some embodiments, the set of one or more HLA-A*31:01 detection reagents comprises a first detection reagent that is specific for the presence of the reference G allele at the locus corresponding to the rs1059457 SNP and is conjugated to a non-nucleotidic detectable moiety, and a second detection reagent that is specific for the presence of the reference G allele at the locus corresponding to the rs79361534 SNP and is conjugated to a non-nucleotidic detectable moiety.

In some embodiments of the kit, the first genomic region does not encompass the locus corresponding to the rs41562315 SNP.

In some embodiments of the kit, the first primer overlaps the locus corresponding to rs41541222 and is specific for the rs41541222 G>T SNP. In some embodiments, the first primer has a nucleotide sequence comprising at least 10 consecutive nucleotides of SEQ ID NO:1. In some embodiments, first primer comprises a nucleic acid sequence of SEQ ID NO:38.

In some embodiments of the kit, the second primer has a nucleotide sequence comprising at least 10 consecutive nucleotides of SEQ ID NO:11. In some embodiments, the second primer comprises a nucleic acid sequence of SEQ ID NO:39.

In some embodiments of the kit, the first set of allele-specific PCR primers consists of the first primer and the second primer.

In some embodiments of the kit, the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP. In some embodiments, the single HLA-A*31:01 detection reagent comprises a nucleic acid sequence of SEQ ID NO:40 or SEQ ID NO:41.

In some embodiments of the kit, each respective detection reagent in the set of one or more HLA-A*31:01 detection reagents is conjugated to one or more detection label. In some embodiments, the one or more detection label comprises a fluorescent dye. In some embodiments, the one or more detection label comprises a matching pair of electronic energy transfer fluorophores comprising a donor fluorophore and a quencher fluorophore for the donor fluorophore.

In some embodiments, the kit further comprises a second set of PCR primers designed to amplify a control genomic locus from the biological sample obtained from the individual and a control detection reagent that binds to the control locus. In some embodiments, the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP, wherein the single HLA-A*31:01 detection reagent is conjugated to a first matching pair of electronic energy transfer fluorophores comprising a first donor fluorophore and a first quencher fluorophore for the first donor fluorophore; and the control detection reagent is conjugated to a second matching pair of electronic energy transfer fluorophores comprising a second donor fluorophore and a second quencher fluorophore for the second donor fluorophore. In some embodiments, the first and the second matching pairs of electronic energy transfer fluorophores are selected from the group consisting of FAM-TAMRA, VIC-TAMRA, TET-TAMRA and JOE-TAMRA, and the first and the second matching pairs of electronic energy transfer fluorophores are different.

### V. BRIEF DESCRIPTION OF THE DRAWINGS

FIG. 1 illustrates exemplary PCR primers and probe for detecting the genomic loci corresponding to SNPs rs1059449, rs41541222, rs1059457, rs79361534, and rs1059471. Presence of the entire set of a rs1059449 G>A SNP, a rs41541222 G>T SNP, a reference allele G at the locus corresponding to rs1059457, a reference allele G at the locus corresponding to rs79361534, and a rs1059471 C>T SNP indicates that an individual carries an HLA-A*31:01 allele.

FIG. 2A-FIG. 2C shows concordance of the results from rs1061235 A>T SNP screening and the improved HLA-A*31:01 screening method described in the current invention with the samples of known HLA genotypes confirmed by NGS.

FIG. 3 shows concordance of the results from three runs in triplicate using the improved HLA-A*31:01 screening method on samples of known HLA genotypes confirmed by NGS.

FIG. 4 shows concordance of the results from the improved HLA-A*31:01 screening method with the samples from Coriell institute and of known HLA genotypes.

FIG. 5A-FIG. 5B shows frequencies of non HLA-A*31 alleles that can be detected by the improved HLA-A*31:01 screening method due to sequence homology with HLA-A*31:01. Among these populations, the total frequency for detection of the non HLA-A*31 alleles among the European Caucasian population in the US is 0.00268%. FIG. 5B shows the results from the improved HLA-A*31:01 screening method on samples containing some of the non HLA-A*31 alleles.

FIG. 6 shows the limit of detection of the improved HLA-A*31:01 screening method. The input DNA of the test samples was serially diluted, and the concordance of the results from the improved HLA-A*31:01 screening method with the samples of known HLA genotypes.

### VI. DETAILED DESCRIPTION

### A. Introduction

This present disclosure provides an improve method for screening a specific HLA allele in an individual. Since HLA alleles have been associated with ADRs developed from various medications, the present disclosure provides a prediction of ADRs in an individual who is in need of a medication, which causes the ADRs associated with a specific HLA allele. As such, by providing an improved method for screening a specific HLA allele, the present disclosure provides a method of treating and clinical support for treating an individual in need of a medication, which causes ADRs associated with the specific HLA allele.

For example, the HLA-A*31:01 allele is generally associated with ADRs developed from carbamazepine, and is commonly screened in an individual who needs carbamazepine. The currently used method for screening an HLA-A*31:01 allele uses detection of an rs1061235 A>T SNP. However, this method gives a high percentage of false positive results, as shown in the present disclosure. As such, the present disclosure provides a more accurate and cost effective method for screening an HLA-A*31:01 allele in an individual who needs carbamazepine, and predicting the likelihood that the individual will develop an adverse reaction towards carbamazepine. This invention also provides an improved method of treating and clinical support for treating an individual in need of carbamazepine.

### B. Definitions

As used herein, the term “biological sample” refers to any sample taken from a subject, which contains nucleic acids reflecting the genotype of the subject with respect to the genomic loci described herein. Examples of biological samples include, but are not limited to, blood samples, saliva samples, buccal cell samples, and the like. A biological sample can be a liquid sample or a solid sample (e.g., a cell or tissue sample). The nucleic acid in the sample can be a cell-free nucleic acid. In some embodiments, a biological sample is processed to extract genomic DNA.

The term “nucleic acid” refers to deoxyribonucleic acid (DNA) and ribonucleic acid (RNA). The term “nucleic acid probe” refers to a single stranded DNA or RNA probe.

As used herein, the term “genomic locus” or “locus” refers to a position (e.g., a site) within a genome, e.g., on a particular chromosome. In some embodiments, a locus refers to a single nucleotide position within a genome, e.g., on a particular chromosome. In some embodiments, a locus refers to a small group of nucleotide positions within a genome. Because normal mammalian cells have diploid genomes, a normal mammalian genome (e.g., a human genome) generally has two copies of every locus on autosomal chromosomes, e.g., one copy on the maternal autosomal chromosome and one copy on the paternal autosomal chromosome.

As used herein, the term “allele” refers to a particular sequence of one or more nucleotides at a genomic locus. Because normal mammalian cells have diploid genomes, a normal mammalian genome (e.g., a human genome) will have two alleles for each genomic locus, which may be the same or different. When a mammal has the same allele at both copies of a locus, they are homozygous for the allele. When a mammal has different alleles at their two copies of a locus, they are heterozygous for the two alleles. In some embodiments, determining the allelic status of a HLA gene in an individual includes determining whether the individual carries, e.g., has at least one copy of, the allele.

As used herein, the term “SNP” or “single nucleotide polymorphism” refers to a substitution of a single nucleotide that occurs at a specific position (e.g., locus) in the genome. A substitution from a first nucleotide X to a second nucleotide Y may be denoted as “X>Y.” For example, a cytosine to thymine SNP may be denoted as “C>T.” Rs number stands for Reference SNP cluster ID, and is an accession number used by researchers and databases to refer to specific SNPs.

### C. Method of Screening for an HLA-A*31:01 Allele

In one aspect, the present disclosure provides a method of screening for an HLA-A*31:01 allele in an individual who is need of carbamazepine, and predicting whether such individual is likely to develop ADRs towards carbamazepine. In general, this method detects five genomic loci corresponding to SNPs rs1059449, rs41541222, rs1059457, rs79361534, and rs1059471. Presence of the entire set of a rs1059449 G>A SNP, a rs41541222 G>T SNP, a reference allele G at the locus corresponding to rs1059457, a reference allele G at the locus corresponding to rs79361534, and a rs1059471 C>T SNP indicates that the individual carries an HLA-A*31:01 allele. Presence of less than the entire set of a rs1059449 G>A SNP, a rs41541222 G>T SNP, a reference allele G at the locus corresponding to rs1059457, a reference allele G at the locus corresponding to rs79361534, and a rs1059471 C>T SNP indicates that the individual does not carry an HLA-A*31:01 allele.

In some embodiments, the method of screening for an HLA-A*31:01 allele does not include detection of a rs41562315 G>A SNP.

In some embodiments, the method of screening for an HLA-A*31:01 allele is combined with detection of a rs1061235 A>T SNP.

1. Assay Design

The method of screening for an HLA-A*31:01 allele includes performing a polymerase chain reaction (PCR) assay designed to amplify a genomic region from a biological sample obtained from an individual, and attempting to detect the amplification of the genomic loci with a set of allele specific nucleic acid probes detectable and capable of specifically binding to the genomic region.

The genomic region to be amplified encompasses the loci corresponding to SNPs rs1059449, rs41541222, rs1059457, rs79361534, and rs1059471. In some embodiments, the genomic region to be amplified does not encompass the locus corresponding to rs41562315.

The screening method for an HLA-A*31:01 allele uses a set of allele specific PCR primers and a set of allele specific nucleic acid probes. The set of PCR primers include a forward primer and a reverse primer flanking the genomic region to be amplified. The set of PCR primers and the set of nucleic acid probes are designed such that they, in combination, (1) overlap with the genomic loci corresponding to SNPs rs1059449, rs41541222, rs1059457, rs79361534, and rs1059471; and (2) are specific for the rs1059449 G>A SNP, rs41541222 G>T SNP, reference allele G at the locus corresponding to rs1059457, reference allele G at the locus corresponding to rs79361534, and rs1059471 C>T SNP. The set of PCR primers and nucleic acid probes are also designed not to bind to the same genomic locus.

In some embodiments, the set of allele specific nucleic acid probe(s) contains only one probe, which is capable of binding to the genomic region to be amplified and specific for at least one of the rs1059449 G>A SNP, rs41541222 G>T SNP, reference allele G at the locus corresponding to rs1059457, reference allele G at the locus corresponding to rs79361534, and rs1059471 C>T SNP. In some embodiments, the set of allele specific nucleic acid probe(s) contains two or more probes, each of which is capable of binding to the genomic region to be amplified and specific for at least one of the rs1059449 G>A SNP, rs41541222 G>T SNP, reference allele G at the locus corresponding to rs1059457, reference allele G at the locus corresponding to rs79361534, and rs1059471 C>T SNP, where each probe does not bind to the same locus.

In some embodiments, the forward primer or the reverse primer overlaps with at least one locus corresponding to the SNPs rs1059449, rs41541222, rs1059457, rs79361534, and rs1059471. In some embodiments, the forward primer overlaps with at least one locus corresponding to the SNPs rs1059449, rs41541222, rs1059457, rs79361534, and rs1059471; and the reverse primer overlaps with at least one locus corresponding to the SNPs rs1059449, rs41541222, rs1059457, rs79361534, and rs1059471, but different from the locus overlapped by the forward primer.

In some embodiments, the forward primer overlaps with the locus corresponding to rs1059449 and is specific for the rs1059449 G>A SNP. The reverse primer overlaps with the locus corresponding to rs1059471 and is specific for the rs1059471 C>T SNP. The set of nucleic acid probes is specific for the rs41541222 G>T SNP, reference allele G at the locus corresponding to rs1059457, and reference allele G at the locus corresponding to rs79361534. In some embodiments, the set of nucleic acid probes contain only one nucleic acid probe, and is for the rs41541222 G>T SNP, reference allele G at the locus corresponding to rs1059457, and reference allele G at the locus corresponding to rs79361534. In some embodiments, the set of nucleic acid probes contains two nucleic acid probes, one being specific for the rs41541222 G>T SNP, and the other being specific the reference allele G at the locus corresponding to rs1059457 and reference allele G at the locus corresponding to rs79361534. In some embodiments, the set of nucleic acid probes contains two nucleic acid probes, one being specific for the rs41541222 G>T SNP and the reference allele G at the locus corresponding to rs1059457, and the other being specific for the reference allele G at the locus corresponding to rs79361534. In some embodiments, the set of nucleic acid probes contains three nucleic acid probes, each one being specific for the rs41541222 G>T SNP, the reference allele G at the locus corresponding to rs1059457, and the reference allele G at the locus corresponding to rs79361534 respectively.

In some embodiments, the forward primer overlaps with the locus corresponding to rs1059449 and is specific for the rs1059449 G>A SNP. The reverse primer overlaps with the loci corresponding to rs79361534 and rs1059471, and is specific for the reference allele G at the locus corresponding to rs79361534 and the rs1059471 C>T SNP. The set of nucleic acid probes are specific for rs41541222 G>T SNP, and the reference allele G at the locus corresponding to rs1059457. In some embodiments, the set of nucleic acid probes contain only one nucleic acid probe. In some embodiments, the set of nucleic acid probes contain two nucleic acid probes, each being specific for the rs41541222 G>T SNP, or the reference allele G at the locus corresponding to rs1059457 respectively.

In some embodiments, the forward primer overlaps with the locus corresponding to rs1059449 and is specific for the rs1059449 G>A SNP. The reverse primer does not overlap with any of the loci corresponding to SNPs rs1059449, rs41541222, rs1059457, rs79361534, and rs1059471. The set of nucleic acid probes are specific for the rs41541222 G>T SNP, reference allele G at the locus corresponding to rs1059457, reference allele G at the locus corresponding to rs79361534, and rs1059471 C>T SNP. In some embodiments, the set of nucleic acid probes contain only one nucleic acid probe. In some embodiments, the set of nucleic acid probes contain two nucleic acid probes, each probe being specific for one or more of the rs41541222 G>T SNP, reference allele G at the locus corresponding to rs1059457, reference allele G at the locus corresponding to rs79361534, and rs1059471 C>T SNP respectively. In some embodiments, the set of nucleic acid probes contain three nucleic acid probes, each probe being specific for one or two of the rs41541222 G>T SNP, reference allele G at the locus corresponding to rs1059457, reference allele G at the locus corresponding to rs79361534, and rs1059471 C>T SNP respectively. In some embodiments, the set of nucleic acid probes contain four nucleic acid probes, each probe being specific for one of the rs41541222 G>T SNP, reference allele G at the locus corresponding to rs1059457, reference allele G at the locus corresponding to rs79361534, and rs1059471 C>T SNP respectively.

In some embodiments, the forward primer overlaps with the loci corresponding to rs1059449 and rs41541222, and the forward primer is specific for the rs1059449 G>A SNP and rs41541222 G>T SNP. The reverse primer overlaps with the loci corresponding to rs79361534 and rs1059471, and is specific for the reference allele G at the locus corresponding to rs79361534 and rs1059471 C>T SNP. The set of nucleic acid probes contains only one probe and is specific for the reference allele G at the locus corresponding to rs1059457.

In some embodiments, the forward primer overlaps with the loci corresponding to rs1059449 and rs41541222, and the forward primer is specific for the rs1059449 G>A SNP and rs41541222 G>T SNP. The reverse primer does not overlap with any of the loci corresponding to SNPs rs1059449, rs41541222, rs1059457, rs79361534, and rs1059471. The set of nucleic acid probes are specific for the reference allele G at the locus corresponding to rs1059457, reference allele G at the locus corresponding to rs79361534, and rs1059471 C>T SNP. In some embodiments, the set of nucleic acid probes contain only one nucleic acid probe. In some embodiments, the set of nucleic acid probes contain two nucleic acid probes, each probe being specific for any one or two of the reference allele G at the locus corresponding to rs1059457, reference allele G at the locus corresponding to rs79361534, and rs1059471 C>T SNP respectively. In some embodiments, the set of nucleic acid probes contain three nucleic acid probes, each probe being specific for one of the reference allele G at the locus corresponding to rs1059457, reference allele G at the locus corresponding to rs79361534, and rs1059471 C>T SNP respectively.

In some embodiments, the forward primer does not overlap with any of the loci corresponding to SNPs rs1059449, rs41541222, rs1059457, rs79361534, and rs1059471. The reverse primer overlaps with the locus corresponding to rs1059471 and is specific for the rs1059471 C>T SNP. The set of nucleic acid probes are specific for the rs1059449 G>A SNP, rs41541222 G>T SNP, reference allele G at the locus corresponding to rs1059457, and reference allele G at the locus corresponding to rs79361534. In some embodiments, the set of nucleic acid probes contain only one nucleic acid probe. In some embodiments, the set of nucleic acid probes contain two nucleic acid probes, each probe being specific for one or more of the rs1059449 G>A SNP, rs41541222 G>T SNP, reference allele G at the locus corresponding to rs1059457, and reference allele G at the locus corresponding to rs79361534 respectively. In some embodiments, the set of nucleic acid probes contain three nucleic acid probes, each probe being specific for one or two of the rs1059449 G>A SNP, rs41541222 G>T SNP, reference allele G at the locus corresponding to rs1059457, and reference allele G at the locus corresponding to rs79361534 respectively. In some embodiments, the set of nucleic acid probes contain four nucleic acid probes, each probe being specific for one of the rs1059449 G>A SNP, rs41541222 G>T SNP, reference allele G at the locus corresponding to rs1059457, and reference allele G at the locus corresponding to rs79361534 respectively.

In some embodiments, the forward primer does not overlap with any of the loci corresponding to SNPs rs1059449, rs41541222, rs1059457, rs79361534, and rs1059471. The reverse primer overlaps with the locus corresponding to rs79361534 and rs1059471, and is specific for the reference allele G at the locus corresponding to rs79361534 and rs1059471 C>T SNP. The set of nucleic acid probes are specific for the rs1059449 G>A SNP, rs41541222 G>T SNP, and reference allele G at the locus corresponding to rs1059457. In some embodiments, the set of nucleic acid probes contains one nucleic acid probe. In some embodiments, the set of nucleic acid probes contains two nucleic acid probes, each probe being specific for one or two of the rs1059449 G>A SNP, rs41541222 G>T SNP, and reference allele G at the locus corresponding to rs1059457 respectively. In some embodiments, the set of nucleic acid probes contains three nucleic acid probes, being probe being specific for one of the rs1059449 G>A SNP, rs41541222 G>T SNP, and reference allele G at the locus corresponding to rs1059457 respectively.

In some embodiments, the forward primer overlaps with the loci corresponding to rs1059449 and rs41541222, and the forward primer is specific for the rs1059449 G>A SNP and rs41541222 G>T SNP. The reverse primer overlaps with the locus corresponding to rs1059471 and is specific for the rs1059471 C>T SNP. The set of nucleic acid probes are specific for the reference allele G at the locus corresponding to rs1059457, and the reference allele G at the locus corresponding to rs79361534. In some embodiments, the set of nucleic acid probes contains only one nucleic acid probe. In some embodiments, the set of nucleic acid probes contains two nucleic acid probes, one probe being specific for the reference allele G at the locus corresponding to rs1059457, and other probe being specific for the reference allele G at the locus corresponding to rs79361534.

Nucleic acid sequences of the PCR primers and probes can be designed according to various embodiments of the method described herein.

For example, in some embodiments, the forward primer overlaps with the loci corresponding to rs1059449 and rs41541222, and is specific for the rs1059449 G>A SNP and rs41541222 G>T SNP. The reverse primer overlaps with the locus corresponding to rs1059471 and is specific for the rs1059471 C>T SNP. The nucleic acid probe is specific for the reference allele G at the locus corresponding to rs1059457, and the reference allele G at the locus corresponding to rs79361534. The forward primer can have a nucleic acid sequence comprising at least 10 consecutive nucleotides of SEQ ID NO: 1 (5′-GAGAGGCCTGAGT-3′), at least 11 consecutive nucleotides of SEQ ID NO: 2 (5′-AGGAGAGGCCTGAGTA-3′), at least 12 consecutive nucleotides of SEQ ID NO: 3 (5′-CAGGAGAGGCCTGAGTAT-3′), at least 13 consecutive nucleotides of SEQ ID NO: 4 (5′-GCAGGAGAGGCCTGAGTATT-3′), at least 14 consecutive nucleotides of SEQ ID NO: 5 (5′-AGCAGGAGAGGCCTGAGTATTG-3′), at least 15 consecutive nucleotides of SEQ ID NO: 6 (5′-GAGCAGGAGAGGCCTGAGTATTGG-3′), at least 16 consecutive nucleotides of SEQ ID NO: 7 (5′-AGAGCAGGAGAGGCCTGAGTATTGGG-3′), at least 17 consecutive nucleotides of SEQ ID NO: 8 (5′-TAGAGCAGGAGAGGCCTGAGTATTGGGA-3′), at least 18 consecutive nucleotides of SEQ ID NO: 9 (5′-ATAGAGCAGGAGAGGCCTGAGTATTGGGAC-3′), or at least 19 consecutive nucleotides of SEQ ID NO: 10 (5′-GATAGAGCAGGAGAGGCCTGAGTATTGGGACC-3′). The reverse primer can have a nucleic acid sequence comprising at least 10 consecutive nucleotides of SEQ ID NO: 11 (5′-ACTCGGTCAATCTGTGAGT-3′), at least 11 consecutive nucleotides of SEQ ID NO: 12 (5′-CACTCGGTCAATCTGTGAGTG-3′), at least 12 consecutive nucleotides of SEQ ID NO: 13 (5′-CCACTCGGTCAATCTGTGAGTGG-3′), at least 13 consecutive nucleotides of SEQ ID NO: 14 (5′-TCCACTCGGTCAATCTGTGAGTGGG-3′), at least 14 consecutive nucleotides of SEQ ID NO: 15 (5′-GTCCACTCGGTCAATCTGTGAGTGGGC-3′), at least 15 consecutive nucleotides of SEQ ID NO: 16 (5′-GGTCCACTCGGTCAATCTGTGAGTGGGCC-3′), at least 16 consecutive nucleotides of SEQ ID NO: 17 (5′-AGGTCCACTCGGTCAATCTGTGAGTGGGCCT-3′), at least 17 consecutive nucleotides of SEQ ID NO: 18 (5′-CAGGTCCACTCGGTCAATCTGTGAGTGGGCCTT-3′), or at least 18 consecutive nucleotides of SEQ ID NO: 19 (5′-CCAGGTCCACTCGGTCAATCTGTGAGTGGGCCTTC-3′). The probe can have a nucleic acid sequence comprising at least 12 consecutive nucleotides of SEQ ID NO: 20

(5′-AGACACGGAATGT-3′)

or SEQ ID NO: 21 (5′-ACATTCCGTGTCT-3′), at least 13 consecutive nucleotides of SEQ ID NO: 22

(5′-GAGACACGGAATGTG-3′)

or SEQ ID NO: 23 (5′-CACATTCCGTGTCTC-3′), at least 14 consecutive nucleotides of SEQ ID NO: 24

(5′-GGAGACACGGAATGTGA-3′)

or SEQ ID NO: 25 (5′-TCACATTCCGTGTCTCC-3′), at least 15 consecutive nucleotides of SEQ ID NO: 26

(5′-AGGAGACACGGAATGTGAA-3′)

or SEQ ID NO: 27 (5′-TTCACATTCCGTGTCTCCT-3′), at least 16 consecutive nucleotides of SEQ ID NO: 28

(5′-CAGGAGACACGGAATGTGAAG-3′)

or SEQ ID NO: 29 (5′-CTTCACATTCCGTGTCTCCTG-3′), at least 17 consecutive nucleotides of SEQ ID NO: 30

(5′-CCAGGAGACACGGAATGTGAAGG-3′)

or SEQ ID NO: 31 (5′-CCTTCACATTCCGTGTCTCCTGG-3′), at least 18 consecutive nucleotides of SEQ ID NO: 32

(5′-GACCAGGAGACACGGAATGTGAAGGC-3′)

or SEQ ID NO: 33 (5′-GCCTTCACATTCCGTGTCTCCTGGTC-3′), at least 19 consecutive nucleotides of SEQ ID NO: 34

(5′-GGACCAGGAGACACGGAATGTGAAGGCC-3′),

or SEQ ID NO: 35 (5′-GGCCTTCACATTCCGTGTCTCCTGGTCC-3′), or at least 20 consecutive nucleotides of SEQ ID NO: 36

(5′-GGGACCAGGAGACACGGAATGTGAAGGCCC-3′)

### or SEQ ID NO: 37 (5′-GGGCCTTCACATTCCGTGTCTCCTGGTCCC-3′).

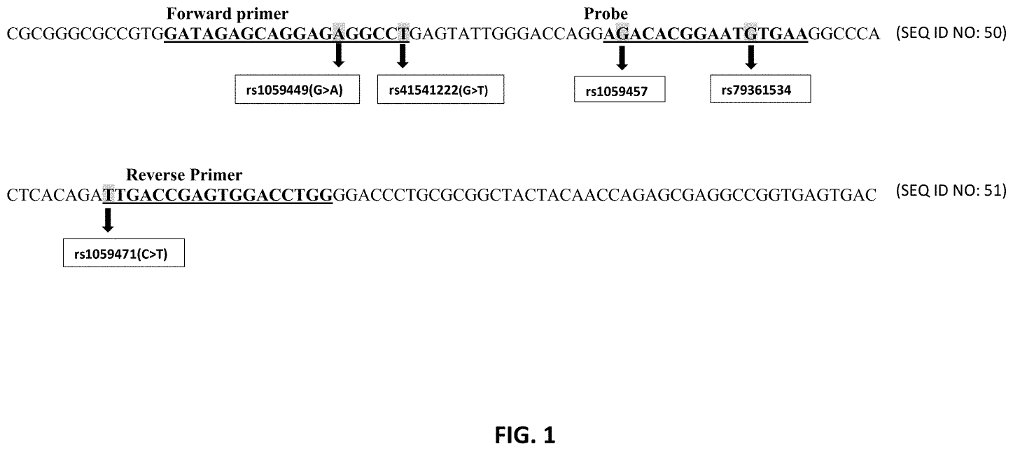

In some embodiments, as shown in FIG. 1, the forward primer comprises a nucleic acid sequence of SEQ ID NO: 38 (5-GATAGAGCAGGAGAGGCCT-3′), the reverse primer comprises a nucleic acid sequence of SEQ ID NO: 39 (5-CCAGGTCCACTCGGTCAA-3′), and the probe comprises a nucleic acid sequence of SEQ ID NO: 40 (5-AGACACGGAATGTGAA-3′) or SEQ ID NO: 41 (5-TTCACATTCCGTGTCT-3′).

The method of screening for an HLA-A*31:01 allele includes performing a PCR assay designed to amplify a genomic region from a biological sample obtained from an individual, and attempting to detect the amplification of the genomic loci with a set of allele specific nucleic acid probes which are detectable and capable of specifically binding to the genomic region.

In some embodiments, the set of allele specific nucleic acid probes are conjugated with one or more detection labels. In some embodiments, each of the set of nucleic acid probe is conjugated to a matching pair of electronic energy transfer fluorophores comprising an excitation fluorophore conjugated to the 5′ end of the nucleic acid probe and a quenching fluorophore for the excitation fluorophore conjugated to the 3′ end of the nucleic acid probe. The quenching fluorophore inhibits the natural fluorescence emission of the excitation fluorophore by fluorescence resonance energy transfer (FRET). Exemplary matching pairs of an excitation and quenching fluorophores include 6-carboxyfluorescein (FAM) and 5-carboxytetramethylrhodamine (TAMRA); tetrachlorofluorescein (TET) and TAMRA; 2′-chloro-7′-phenyl-1,4-dichloro-6-carboxyfluorescein (VIC) and TAMRA; and 5′-Dichloro-dimethoxy-fluorescein (JOE) and TAMRA; FAM and Black Hole Quencher 1 (BHQ-1), JOE and BHQ-1; TET and BHQ-1; Cyanine 3 and BHQ-2, Texas Red and BHQ-2; and Cyanine 3 and BHQ-3. The PCR primers and the set of dual labeled nucleic acid probes are added together to the PCR assay. During the PCR, the forward and reverse primers and the set of probes anneal specifically to the genomic region encompassing the loci corresponding to SNPs rs1059449, rs41541222, rs1059457, rs79361534, and rs1059471. The signal from the fluorophore on the 5′ end of the probe is quenched by the fluorophore on the 3′ end. DNA polymerase then carries out the extension of the primers and replicates the template to which the set of probes is bound. The 5′ exonuclease activity of the polymerase cleaves the probes, releasing the fluorophore on the 5′ end away from the close vicinity of the quencher on the 3′ end. With each cycle of PCR, more fluorophores from the 5′ end are released, resulting in an increase in fluorescence intensity proportional to the amount of genomic region synthesized. As such, if the individual carries the rs1059449 G>A SNP, rs41541222 G>T SNP, reference allele G at the locus corresponding to rs1059457, reference allele G at the locus corresponding to rs79361534, and rs1059471 C>T SNP, an increase in the intensity of the 5′ end fluorophore will be detected, and the individual is deemed to be a carrier of the HLA-A*31:01 allele.

For example, the method of screening for an HLA-A*31:01 allele can be performed with a PCR assay comprising a forward primer (e.g., SEQ ID NO: 38), a reverse primer (e.g., SEQ ID NO: 39) and a probe labeled with VIC on the 5′ end and TAMRA on its 3′ end (e.g., SEQ ID NO: 40 or SEQ ID NO: 41). The VIC fluorescence signal is quantified in real time. A steady increase of the VIC fluorescence signal indicates that the genomic region encompasses the rs1059449 G>A SNP, rs41541222 G>T SNP, reference allele G at the locus corresponding to rs1059457, reference allele G at the locus corresponding to rs79361534, and rs1059471 C>T SNP. As such, and the individual is deemed to be a carrier of the HLA-A*31:01 allele. Absence of VIC fluorescence signal indicates that the genomic region does not encompasses the entire set of the rs1059449 G>A SNP, rs41541222 G>T SNP, reference allele G at the locus corresponding to rs1059457, reference allele G at the locus corresponding to rs79361534, and rs1059471 C>T SNP. As such, and the individual is not deemed to be a carrier of the HLA-A*31:01 allele.

In some embodiments, the PCR assay is a multiplex PCR assay where a second set of PCR primers and probe are used to bind and amplify specifically a control genomic locus, such as beta-actin, globin, or tubulin. The probe for the control genomic locus is labeled with a different pair of electronic energy transfer fluorophores compared to the probe used for the detecting the HLA-A*31:01 allele, so that the fluorescence signals from the two sets of probes are distinguished. For example, as shown in Example 2, primers and a probe amplifying beta-actin (e.g., a forward primer comprising a nucleic acid sequence of SEQ ID NO: 42, a reverse primer comprising a nucleic acid sequence of SEQ ID NO: 43 and a probe comprising a nucleic acid sequence of SEQ ID NO: 44 or SEQ ID NO: 45 labeled with FAM on the 5′ end and TAMRA on the 3′ end) can be used in the multiplex PCR assay together with the primers and set of probes used to detect the HLA-A*31:01 allele described herein.

In some embodiments, the method of screening for an HLA-A*31:01 allele comprises performing a PCR assay to amplify a genomic region encompassing loci corresponding to rs1059449, rs41541222, rs1059457, rs79361534, and rs1059471 using the forward and reverse primers described herein; and hybridization of the amplified genomic region using the set of nucleic acid probes described herein. The set of nucleic acid probes is conjugated with one or more detection labels. Various methods of nucleic acid hybridization and detection can be used, such as southern blotting and array based detection method where, for example, the labeled probes are immobilized onto a solid support capturing the amplified genomic DNA. Various types of detection labels can be conjugated to the set of nucleic acid probes, such as a fluorophore (e.g., a rhodamine, a fluorescein, a cyanine, a ATTO dye, an Alexa Fluor dyes and a derivative of any of the above fluorphores), a radioactive label (e.g., ³²P, ³³P and ³⁵S), or a hapten (e.g., biotin, digoxigenin, and dinitrophenyl). A fluorophore labeled probe and radioactive labeled probe can be detected directly. A hapten labeled probe can be detected indirectly. For example, a biotinylated probe can be detected through streptavidin conjugated to a reporter enzyme (peroxidase or alkaline phosphatase) or a fluorescent dye. A digoxigenin labeled probe can be detected by digoxigenin antibody conjugated with a reporter enzyme (peroxidase or alkaline phosphatase) or a fluorescent dye. If the set of nucleic acid probes is detected after hybridization, the individual is deemed to be a carrier of the HLA-A*31:01 allele. If one or more of the set of nucleic acid probes is not detected, the individual is not deemed to be a carrier of the HLA-A*31:01 allele.

2. Combination with Rs1061235 Detection

In another aspect of the present disclosure, the method of screening for an HLA-A*31:01 allele described above is combined with an assay designed to detect the rs1061235 A>T SNP for determining whether an individual is carrying an HLA-A*31:01 allele. The method of screening for an HLA-A*31:01 allele and the assay for detecting the rs1061235 A>T SNP can be performed in parallel or sequentially.

In some embodiments, an individual is screened for the rs1061235 A>T SNP first. If the individual does not carry the rs1061235 A>T SNP, no further assay is required and the individual is deemed to not carry the HLA-A*31:01 allele. If the individual carries the rs1061235 A>T SNP, the individual is further tested for the presence of the rs1059449 G>A SNP, rs41541222 G>T SNP, reference allele G at the locus corresponding to rs1059457, reference allele G at the locus corresponding to rs79361534, and rs1059471 C>T SNP using the method described herein. This is due to a high percentage of false positive results associated with the rs1061235 A>T SNP, as shown in Example 1.

In some embodiments, an individual is tested for the rs1061235 A>T SNP in parallel with the method described herein for detecting the genomic loci corresponding to rs1059449, rs41541222, rs1059457, rs79361534, and rs1059471. If the individual carries all of the rs1061235 A>T SNP, rs1059449 G>A SNP, rs41541222 G>T SNP, reference allele G at the locus corresponding to rs1059457, reference allele G at the locus corresponding to rs79361534, and rs1059471 C>T SNP, the individual is deemed to carry the HLA-A*31:01 allele. If the individual does not carry any one of the entire set of the rs1061235 A>T SNP, rs1059449 G>A SNP, rs41541222 G>T SNP, reference allele G at the locus corresponding to rs1059457, reference allele G at the locus corresponding to rs79361534, and rs1059471 C>T SNP, the individual is deemed not carry the HLA-A*31:01 allele.

In some embodiments, an individual is tested first for the presence of the rs1059449 G>A SNP, rs41541222 G>T SNP, reference allele G at the locus corresponding to rs1059457, reference allele G at the locus corresponding to rs79361534, and rs1059471 C>T SNP using the method described herein, followed by detection of the rs1061235 A>T SNP. If the individual carries all of the rs1061235 A>T SNP, rs1059449 G>A SNP, rs41541222 G>T SNP, reference allele G at the locus corresponding to rs1059457, reference allele G at the locus corresponding to rs79361534, and rs1059471 C>T SNP, the individual is deemed to carry the HLA-A*31:01 allele. If the individual does not carry any one of the entire set of the rs1061235 A>T SNP, rs1059449 G>A SNP, rs41541222 G>T SNP, reference allele G at the locus corresponding to rs1059457, reference allele G at the locus corresponding to rs79361534, and rs1059471 C>T SNP, the individual is deemed not carry the HLA-A*31:01 allele.

Various methods can be used to assay whether an individual carries the rs1061235 A>T SNP. For example, a set of allele specific primers can be designed to specifically bind and amplify the genomic locus encompassing the rs1061235 using PCR assay. Either the forward primer or the reverse primer can be designed to overlap with the locus corresponding to the rs1061235, and be specific for rs1061235 A>T SNP. A PCR assay (e.g., a real time PCR) can be carried out using the primers for detecting the rs1061235 A>T SNP. In some embodiments, the PCR assay can also include a second set of PCR primers which bind and amplify a control genomic locus, such as actin, globin, or tubulin. For example, PCR primers for amplifying beta-actin can comprise nucleic acid sequences of SEQ ID NO: 42 and SEQ ID NO: 43 respectively.

Alternatively, a pair of forward and reverse PCR primers can be designed to amplify a genomic region encompassing the locus corresponding to the rs1061235. An allele specific probe can be designed to bind specifically to the rs1061235 A>T SNP. For example, a forward primer can comprise a nucleic acid sequence of SEQ ID NO: 46 (5′-CTGAGAGGCAAGAGTTGTTCC-3′), a reverse primer can comprise a nucleic acid sequence of SEQ ID NO: 47 (5′-GCAGGTGCCTTTGCAGAAA-3′), and a probe can comprise a nucleic acid sequence of SEQ ID NO: 48 (5′-CTGNCCTTCCCTTTGTGACTTGAAGAACCCTGACTTTGTTTCTGCAAAGGC-3′, SEQ ID NO: 49 (5′-GCCTTTGCAGAAACAAAGTCAGGGTACTTCAAGTCACAAAGGGAAGGNCAG-3′),

SEQ ID NO: 52 (5′-CTGNCCTTCCCTTTGTGACTTGAAGTACCCTGACTTTGTTTCTGCAAAGGC-3′ or SEQ ID NO: 53 (5′-GCCTTTGCAGAAACAAAGTCAGGGTTCTTCAAGTCACAAAGGGAAGGNCAG-3′).

In some embodiment, the probe specific for the rs1061235 A>T SNP is conjugated to a matching pair of electronic energy transfer fluorophores comprising an excitation fluorophore conjugated to the 5′ end of the nucleic acid probe and a quenching fluorophore for the excitation fluorophore conjugated to the 3′ end of the nucleic acid probe as described herein. Exemplary matching pairs of an excitation and quenching fluorophores include 6-carboxyfluorescein (FAM) and 5-carboxytetramethylrhodamine (TAMRA); tetrachlorofluorescein (TET) and TAMRA; 2′-chloro-7′-phenyl-1,4-dichloro-6-carboxyfluorescein (VIC) and TAMRA; and 5′-Dichloro-dimethoxy-fluorescein (JOE) and TAMRA; FAM and Black Hole Quencher 1 (BHQ-1), JOE and BHQ-1; TET and BHQ-1; Cyanine 3 and BHQ-2, Texas Red and BHQ-2; and Cyanine 3 and BHQ-3. PCR primers and a dual labeled nucleic acid probe are added together to a PCR assay for detecting the rs1061235 A>T SNP. The PCR assay can also include a second pair of PCR primers and a second probe which bind and detect a control genomic locus, such as actin, globin, or tubulin.

In some other embodiments, the probe specific for the rs1061235 A>T SNP is conjugated to a fluorophore (e.g., a rhodamine, a fluorescein, a cyanine, a ATTO dye, an Alexa Fluor dyes or a derivative of any of the above fluorphores), a radioactive label (e.g., ³²P, ³³P or ³⁵S), or a hapten (e.g., biotin, digoxigenin, or dinitrophenyl). Accordingly, a PCR assay is carried out first to amplify the genomic region encompassing the genomic locus corresponding to the rs1061235. The amplified genomic region is then hybridized with the labeled probe for detecting the presence or absence of the rs1061235 A>T SNP.

### D. Treatment of Patients in Need of Carbamazepine

In another aspect, the present disclosure is related to a method of treating an individual who needs carbamazepine and a method of providing clinical support for the treatment of an individual who needs carbamazepine. The individual may be diagnosed with or suffer a condition such as epilepsy, trigeminal neuralgia, acute manic and mixed episodes in bipolar I disorder, neuropathic pains, fibromyalgia, or refractory schizophrenia.

To treat an individual who needs carbamazepine, the individual is first screened to determine whether the individual is a carrier of the HLA-A*31:01 allele. The screening is based on determining whether the individual carries the rs1059449 G>A SNP, rs41541222 G>T SNP, reference G allele at the locus corresponding to the rs1059457 SNP, reference G allele at the locus corresponding to the rs79361534 SNP, and rs1059471 C>T SNP. If the individual carries all of the rs1059449 G>A SNP, rs41541222 G>T SNP, reference allele G at the locus corresponding to rs1059457, reference allele G at the locus corresponding to rs79361534, and rs1059471 C>T SNP, the individual is deemed to carry the HLA-A*31:01 allele and likely to develop ADRs towards carbamazepine. As such, the individual is administered with an alternative medication to carbamazepine. If the individual does not carry all of the rs1059449 G>A SNP, rs41541222 G>T SNP, reference allele G at the locus corresponding to rs1059457, reference allele G at the locus corresponding to rs79361534, and rs1059471 C>T SNP are detected in the individual, the individual is deemed not to carry the HLA-A*31:01 allele and not likely to develop ADRs towards carbamazepine. As such, the individual is administered with carbamazepine. In some embodiments, the screening does not include analysis of the locus corresponding to rs41562315.

To provide clinical support for the treatment of an individual who needs carbamazepine, the individual is first screened to determine whether the individual is a carrier of the HLA-A*31:01 allele. The screening is based on determining whether the individual carries the rs1059449 G>A SNP, rs41541222 G>T SNP, reference G allele at the locus corresponding to the rs1059457 SNP, reference G allele at the locus corresponding to the rs79361534 SNP, and rs1059471 C>T SNP. Based on the screening result, a report is transmitted to a health care professional recommending one or more possible treatment regimens for the individual. If individual carries all of the rs1059449 G>A SNP, rs41541222 G>T SNP, reference allele G at the locus corresponding to rs1059457, reference allele G at the locus corresponding to rs79361534, and rs1059471 C>T SNP, the individual is deemed to carry the HLA-A*31:01 allele. As a result, the one or more possible treatment regimens recommended in the report does not include administration of carbamazepine to the individual. If the individual does not carry all of the rs1059449 G>A SNP, rs41541222 G>T SNP, reference allele G at the locus corresponding to rs1059457, reference allele G at the locus corresponding to rs79361534, and rs1059471 C>T SNP, the individual is deemed not to carry the HLA-A*31:01 allele. As a result, the one or more possible treatment regimens recommended in the report include administration of carbamazepine to the individual. In some embodiments, the screening does not include analysis of the locus corresponding to rs41562315.

In some embodiments, to determine whether the individual carries the rs1059449 G>A SNP, rs41541222 G>T SNP, reference G allele at the locus corresponding to the rs1059457 SNP, reference G allele at the locus corresponding to the rs79361534 SNP, and rs1059471 C>T SNP, a PCR assay is performed on a biological sample obtained from the individual to amplify a genomic region encompassing the loci corresponding to SNPs rs1059449, rs41541222, rs1059457, rs79361534, and rs1059471. For example, the PCR assay described herein can be used, including the allele specific primers and probes described herein.

In some embodiments, the method of treating an individual who needs carbamazepine further includes screening for the rs1061235 A>T SNP, for example using the method described herein. If the individual carries all of the rs1059449 G>A SNP, rs41541222 G>T SNP, reference allele G at the locus corresponding to rs1059457, reference allele G at the locus corresponding to rs79361534, rs1059471 C>T SNP, and rs1061235 A>T SNP, the individual is deemed to carry the HLA-A*31:01 allele and likely to develop ADRs towards carbamazepine. As such, the individual is administered with an alternative medication to carbamazepine. If the individual does not carry all of the rs1059449 G>A SNP, rs41541222 G>T SNP, reference allele G at the locus corresponding to rs1059457, reference allele G at the locus corresponding to rs79361534, rs1059471 C>T SNP and rs1061235 A>T SNP, the individual is deemed not to carry the HLA-A*31:01 allele and not likely to develop ADRs towards carbamazepine. As such, the individual is administered with carbamazepine.

In some embodiments, the method of providing clinical support for the treatment of an individual who needs carbamazepine further includes screening for the rs1061235 A>T SNP, for example using the method described herein. If individual carries all of the rs1059449 G>A SNP, rs41541222 G>T SNP, reference allele G at the locus corresponding to rs1059457, reference allele G at the locus corresponding to rs79361534, rs1059471 C>T SNP and rs1061235 A>T SNP, the individual is deemed to carry the HLA-A*31:01 allele. As a result, the one or more possible treatment regimens recommended in the report does not include administration of carbamazepine to the individual. If the individual does not carry all of the rs1059449 G>A SNP, rs41541222 G>T SNP, reference allele G at the locus corresponding to rs1059457, reference allele G at the locus corresponding to rs79361534, rs1059471 C>T SNP, and rs1061235 A>T SNP, the individual is deemed not to carry the HLA-A*31:01 allele. As a result, the one or more possible treatment regimens recommended in the report include administration of carbamazepine to the individual.

### E. Screening a Specific HLA Allele

Another aspect of the present disclosure is related to a method for screening a specific HLA allele in an individual, including, but not limited to, a specific HLA allele associated with ADRs, such as HLA-C*03:02 which is associated with ADRs, for example, from allopurinol, HLA-B*13:01 which is associated with ADRs, for example, from dapsone; HLA-B*15:02 which is associated with ADRs, for example, from carbamazepine and phenytoin; HLA-B*15:11 which is associated with ADRs, for example, from carbamazepine; HLA-B*35:01 which is associated with ADRs, for example, from nevirapine; HLA-B*38:02 which is associated with ADRs, for example, from carbimazole and methimazole; HLA-B*40:01 which is associated with ADRs, for example, from carbamazepine; HLA-B*57:01 associated with ADRs, for example, from abacavir; HLA-B*59:01 associated with ADRs, for example, from methazo, HLA-C*01:02 associated with ADRs, for example, from methazolamide; HLA-DRB1*01:01 associated with ADRs, for example, from nevirapine; and HLA-DRB1*07:01 associated with ADRs, for example, from lapatinib.

The method of screening a specific HLA allele in an individual is based on a real time PCR assay that detects the nucleotides corresponding to a set of two or more genomic loci (e.g., two, three, four, five, six, seven, eight, nine, ten or more), where the nucleotides corresponding to the set of two or more genomic loci distinguish the specific HLA allele from other HLA alleles. The PCR assay comprises a set of allele specific forward and reverse primers and a set of allele specific nucleic acid probes (e.g., one or more, two or more, three or more probes) conjugated to a matching pair of electronic energy transfer fluorophores comprising an excitation (donor) fluorophore and a quenching fluorophore for the excitation fluorophore. The forward and reverse primers flank a genomic region encompassing the set of two or more genomic loci, and the set of probes is capable of binding specifically to the genomic region and overlaps with at least one of the two or more genomic loci not overlapped by the PCR primers. The forward and reverse primers and the set of probes in combination overlap with the entire set of two or more genomic loci, and are specific for the nucleotides corresponding to the set of the genomic loci that distinguish the specific HLA allele from other HLA alleles. The PCR primers and set of nucleic acid probes are mixed before the real time PCR assay starts, and detection of the entire set of nucleic acid probes during the PCR assay indicates that all the nucleotides which distinguish the specific HLA allele from other HLA alleles are present in the individual. As such, the individual is deemed to carry out the specific HLA allele. If less that the entire set of nucleic acid probes is detected, this indicates that not all of the nucleotides which distinguish the specific HLA allele from other HLA alleles are present in the individual. As such, the individual is deemed not to carry out the specific HLA allele.

In some embodiments, either the forward primer or the reverse primer overlaps with at least one of the two or more genomic loci. In some embodiments, when the set of two or more genomic loci comprises three or more loci, the forward primer and the reverse primer each overlaps with at least one of the genomic loci.

In some embodiments, the set of nucleic acid probes contains a single probe overlapping with at least one locus from the set of two or more genomic loci. In some embodiments, when the set of two or more genomic loci contains three or more loci, the set of nucleic acid probes contains two probes, and each of the probes overlaps with at least one of the genomic loci. In some embodiments, when set of two or more genomic loci contains four or more loci, the set of nucleic acid probes contains three or more probes, and each of the probes overlaps with at least one of the genomic loci.

Each probe of the entire set of one or more nucleic acid probe is conjugated with a matching pair of electronic energy transfer fluorophores comprises an excitation (donor) fluorophore conjugated to the 5′ end of the nucleic acid probe and a quenching fluorophore for the excitation fluorophore conjugated to the 3′ end of the nucleic acid probe. The quenching fluorophore inhibits the natural fluorescence emission of the excitation fluorophore by fluorescence resonance energy transfer (FRET). Exemplary matching pairs of an excitation and quenching fluorophores include 6-carboxyfluorescein (FAM) and 5-carboxytetramethylrhodamine (TAMRA); tetrachlorofluorescein (TET) and TAMRA; 2′-chloro-7′-phenyl-1,4-dichloro-6-carboxyfluorescein (VIC) and TAMRA; and 5′-Dichloro-dimethoxy-fluorescein (JOE) and TAMRA; FAM and Black Hole Quencher 1 (BHQ-1), JOE and BHQ-1; TET and BHQ-1; Cyanine 3 and BHQ-2, Texas Red and BHQ-2; and Cyanine 3 and BHQ-3. The PCR primers and the set of dual labeled nucleic acid probes are added together to the PCR assay. During the PCR, the forward and reverse primers and the set of probes anneal specifically to the genomic region encompassing the loci corresponding to the set of two or more SNPs. The signal from the fluorophore on the 5′ end of the probe is quenched by the fluorophore on the 3′ end. DNA polymerase then carries out the extension of the primers and replicates the template to which the set of probes is bound. The 5′ exonuclease activity of the polymerase cleaves the probes, releasing the fluorophore on the 5′ end away from the close vicinity of the quencher on the 3′ end. With each cycle of PCR, more donor fluorophores from the 5′ end are released, resulting in an increase in the donor fluorescence signal proportional to the amount of genomic region synthesized. As such, when the donor fluorescence signal(s) from the entire set of nucleic acid probes is detected, the individual is deemed to be a carrier of the specific allele of the HLA gene. When the donor fluorescence signal(s) from less than the entire set of one or more nucleic acid probes is detected, the individual is deemed not to be a carrier of the specific allele of the HLA gene.

In some embodiments, the real time PCR assay is a multiplex PCR assay where a second set of PCR primers and labeled probe is used to bind and amplify specifically a control genomic locus, such as actin, globin, or tubulin. The probe for the control genomic locus is labeled with a different pair of electronic energy transfer fluorophores compared to those conjugated on the set of probes for detecting the specific HLA allele, so that the fluorescence signals from the different set of probes are distinguished.

Furthermore, the real time PCR assay can be combined with detection of one or more SNPs located in a genomic region that is not flanked by the set of primers used in the real time PCR assay described herein. The one or more SNPs are genetically associated with the specific HLA allele. In some embodiments, the one or more SNPs are located in one or more genomic regions outside of the HLA gene.

In some embodiments, the method for screening a specific HLA allele in an individual described herein is used to predict whether such individual will develop ADRs from a medication. In some embodiments, the method for screening a specific HLA allele in an individual described herein is used to provide treatment to such individual who needs a medication, and the medication can result in ADRs in an individual carrying the specific HLA allele. If the individual is deemed to carry the specific HLA allele, the medication will be administered. If the individual is deemed to carry the specific HLA allele, the medication will not be administered to the individual.

In some embodiments, the method for screening a specific HLA allele described herein is used to provide clinical support for treatment of an individual who needs a medication, and the medication can result in ADRs in an individual carrying the specific HLA allele. If the individual is deemed to carry the specific HLA allele, the individual is recommended to be administered with an alternative to the medication. If the individual is deemed to not to carry the specific HLA allele, the individual is recommended to be administered with the medication.

F Kit

Another aspect of the present disclosure provides a kit comprising the PCR primers and nucleic acid probe(s) for screening a specific HLA allele described herein.

In some embodiments, the present disclosure provides a kit comprising a set of PCR primers and the set of nucleic acid probes described herein for screening an HLA-A*31:01 allele. In some embodiments, the set of probes in the kit is labeled with a matching pair of electronic energy transfer fluorophores comprising a first donor fluorophore and a first quencher fluorophore for the first donor fluorophore as described herein. In some embodiments, the set of probes in the kit is conjugated to a fluorophore, a radioactive label, or a hapten as described herein.

A kit for screening an HLA-A*31:01 allele can include the set of primers and probes described herein. For example, the kit for screening an HLA-A*31:01 allele can include a forward primer comprising a nucleic acid sequence of SEQ ID NO: 38, a reverse primer comprising a nucleic acid sequence of SEQ ID NO: 39, and a probe comprising a nucleic acid sequence of SEQ ID NO: 40 or SEQ ID NO: 41. The probe is conjugated with a matching pair of electronic energy transfer fluorophores such as a pair of FAM-TAMRA, VIC-TAMRA, TET-TAMRA or JOE-TAMRA.

For example, the kit for screening an HLA-A*31:01 allele can include a forward primer selected from a group consisting of nucleic acid sequences comprising at least 10 consecutive nucleotides of SEQ ID NO: 1, at least 11 consecutive nucleotides of SEQ ID NO: 2, at least 12 consecutive nucleotides of SEQ ID NO: 3, at least 13 consecutive nucleotides of SEQ ID NO: 4, at least 14 consecutive nucleotides of SEQ ID NO: 5, at least 15 consecutive nucleotides of SEQ ID NO: 6, at least 16 consecutive nucleotides of SEQ ID NO: 7, at least 17 consecutive nucleotides of SEQ ID NO: 8, at least 18 consecutive nucleotides of SEQ ID NO: 9, or at least 19 consecutive nucleotides of SEQ ID NO: 10; a reverse primer selected from a group consisting of nucleic acid sequences comprising at least 10 consecutive nucleotides of SEQ ID NO: 11, at least 11 consecutive nucleotides of SEQ ID NO: 12, at least 12 consecutive nucleotides of SEQ ID NO: 13, at least 13 consecutive nucleotides of SEQ ID NO: 14, at least 14 consecutive nucleotides of SEQ ID NO: 15, at least 15 consecutive nucleotides of SEQ ID NO: 16, at least 16 consecutive nucleotides of SEQ ID NO: 17, at least 17 consecutive nucleotides of SEQ ID NO: 18, or at least 18 consecutive nucleotides of SEQ ID NO: 19; and a probe selected from a group consisting of nucleic acid sequences comprising at least 12 consecutive nucleotides of SEQ ID NOs: 20 or 21, at least 13 consecutive nucleotides of SEQ ID NOs: 22 or 23, at least 14 consecutive nucleotides of SEQ ID NOs: 24 or 25, at least 15 consecutive nucleotides of SEQ ID NOs: 26 or 27, at least 16 consecutive nucleotides of SEQ ID NOs: 28 or 29, at least 17 consecutive nucleotides of SEQ ID NOs: 30 or 31, at least 18 consecutive nucleotides of SEQ ID NOs: 32 or 33, at least 19 consecutive nucleotides of SEQ ID NOs: 34 or 35, or at least 20 consecutive nucleotides of SEQ ID NOs: 36 or 37. In some embodiments, the probe is selected from the reverse complete sequences of the group consisting of nucleic acid sequences comprising at least 12 consecutive nucleotides of SEQ ID NO: 20, at least 13 consecutive nucleotides of SEQ ID NO: 21, at least 14 consecutive nucleotides of SEQ ID NO: 22, at least 15 consecutive nucleotides of SEQ ID NO: 23, at least 16 consecutive nucleotides of SEQ ID NO: 24, at least 17 consecutive nucleotides of SEQ ID NO: 25, at least 18 consecutive nucleotides of SEQ ID NO: 26, at least 19 consecutive nucleotides of SEQ ID NO: 27, or at least 20 consecutive nucleotides of SEQ ID NO: 28.

In some embodiments, the kit for screening an HLA-A*31:01 allele further comprises primers and optional probes for amplifying a control genomic locus, such as actin, globin, or tubulin as described herein. For example, the kit can further include a forward primer comprising a nucleic acid sequence of SEQ ID NO: 42, a reverse primer comprising a nucleic acid sequence of SEQ ID NO: 43, and a probe comprising a nucleic acid sequence of SEQ ID NO: 44 or SEQ ID NO: 45.

In some embodiments, the kit for screening an HLA-A*31:01 allele further comprises reagents for detecting rs1061235 A>T SNP. For example, the kit can further include primers and optional probes for detecting the rs1061235 A>T SNP as described herein. In some embodiments, the kit for screening an HLA-A*31:01 allele further includes a forward primer comprising a nucleic acid sequence of SEQ ID NO: 46, a reverse primer comprising a nucleic acid sequence of SEQ ID NO: 47, and a probe comprising a nucleic acid sequence of SEQ ID NO: 48, SEQ ID NO: 49, SEQ ID NO: 52, or SEQ ID NO: 53. In some embodiments, the probe for detecting the rs1061235 A>T SNP is conjugated with a detection label, such as a matching pair of excitation fluorophore and quenching fluorophore, a fluorophore, a radioactive label, or a hapten described herein.

### F. EXAMPLES

The invention now being generally described, will be more readily understood by reference to the following examples, which are included merely for purposes of illustration of certain aspects and embodiments of the present invention, and is not intended to limit the invention.

### Example 1: Rs1061235 Screening Gave False Positive Results

Rs1061235 A>T SNP has been widely used for screening individuals as an indicator for the presence of an HLA-A*31:01 allele and for determining the risk of an individual developing ADRs before the treatment with carbamazepine. Here, the accuracy of using rs1061235 A>T SNP as an indicator for the presence of an HLA-A*31:01 allele was examined.

Buccal swabs from individuals were collected remotely (physicians' offices, institutions, hospitals) according to the instructions from the collection kit. The samples were transported to the laboratory by commercial carrier at ambient temperature. Two swab specimens are collected from each individual: one swab was used in the DNA isolation procedure; the second swab was held in reserve in case of processing mishap or batch failure. The wood swabs are stable at room temperature for at least one year. DNA was then extracted from the buccal epithelial cells from each sample using MagMAX 96 DNA Multi Sample Kit according the manufacturer's instructions. Typically, approximately 2-12 μg of DNA was obtained from each sample.

The rs1061235 A>T SNP was screened in each sample by real time PCR using OpenArray plate technology on the QuantStudios 12 k Flex system. Forwards primer is 5′-CTGAGAGGCAAGAGTTGTTCCT-3′ (SEQ ID NO: 46), and reverse primer is 5′-GCAGGTGCCTTTGCAGAAA-3′ (SEQ ID NO:47), and the probe is 5′-CTGNCCTTCCCTTTGTGACTTGAAGTACCCTGACTTTGTTTCTGCAAAGGC-3′ (SEQ ID NO:48). The samples tested for rs1061235 A>T SNP were also sent to ProImmune for HLA typing using next generation sequencing (NGS).

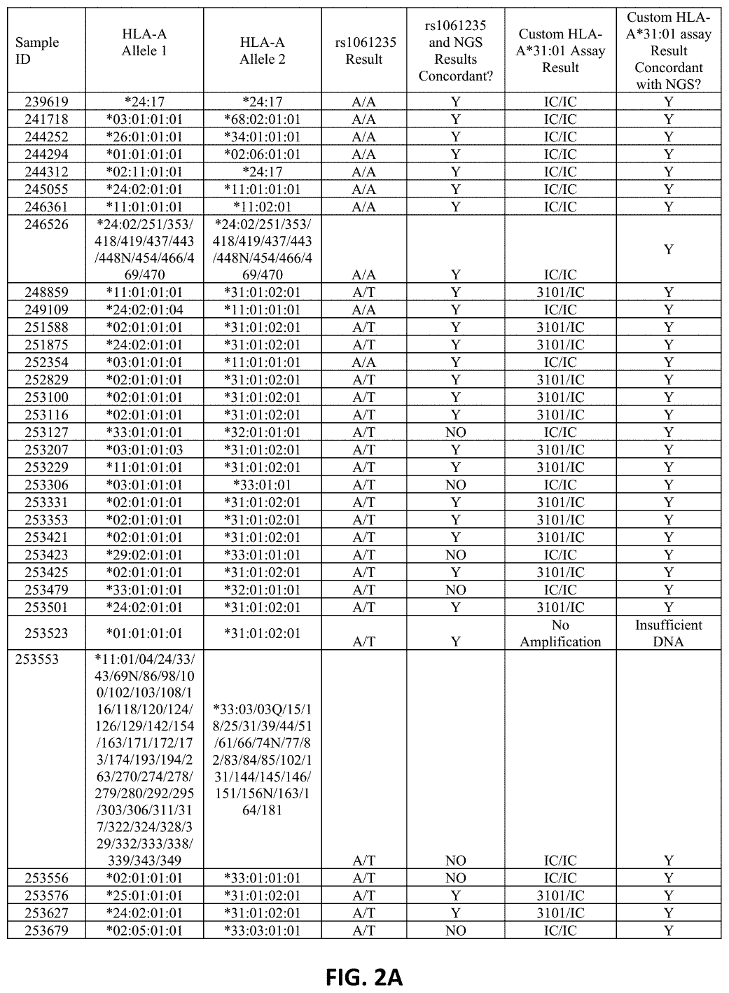

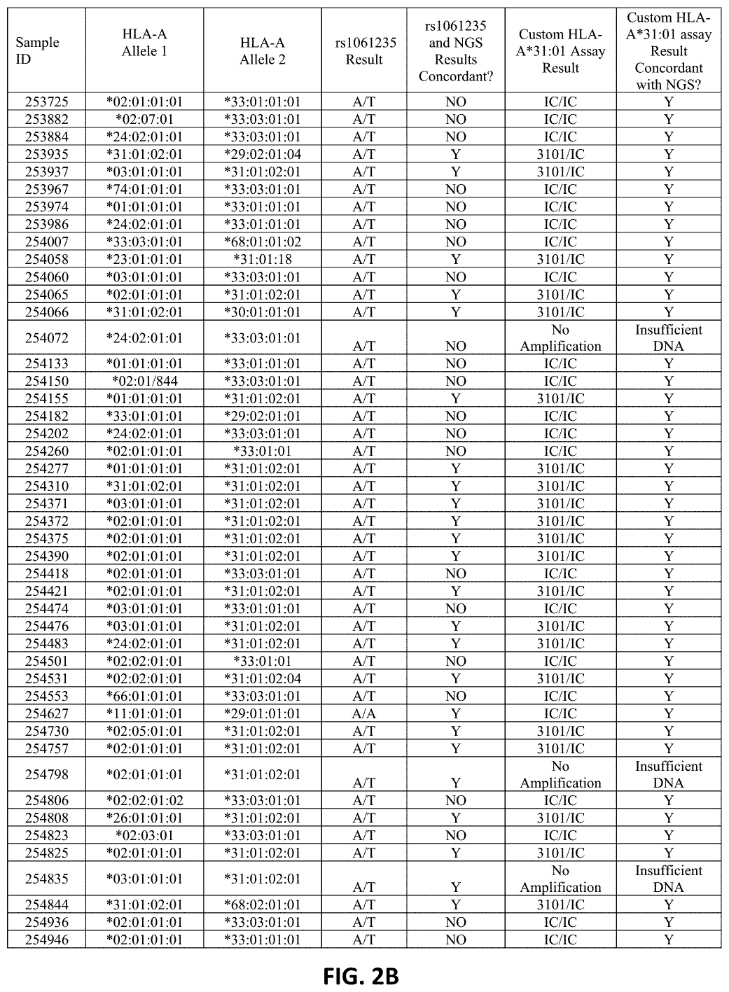

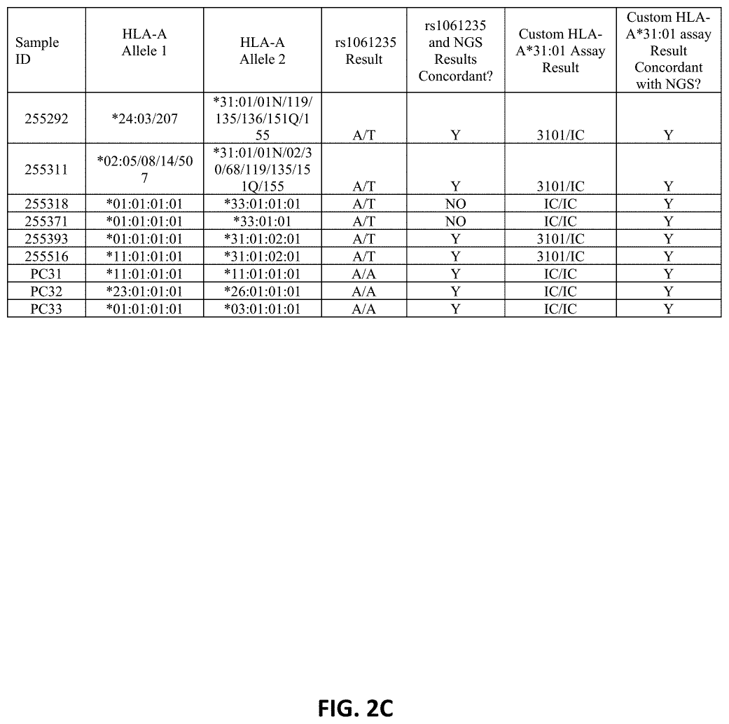

As shown in FIG. 2, among the 93 samples tested, rs1061235 A>T SNP was detected in 79 samples. However, among these 79 samples, 47 carried an HLA-A*31:01 allele, and the other 32 samples carried alleles other than HLA-A*31:01 (e.g., most notably HLA-A*33:01 and *33:03 alleles). This result indicates a high false positive rate (around 41%) in the rs1061235 A>T SNP screening assay for an HLA-A*31:01 allele.

### Example 2 Improved Method of Screening an HLA-A*31:01 Allele

(1) Identification of SNPs for Screening an HLA-A*31:01 Allele

Because carbamazepine and certain other related drugs have been known to cause adverse drug reactions in certain populations, it is important to establish a more accurate assay for screening the HLA-A*31:01 genotype.

Genomic sequences of the HLA-A gene were analyzed from the data published in various sources, including the 1000 Genomes Project; Solberg et al. (2008) Human Immunology 69(7):443-64, where HLA alleles were collected from 497 population samples; datasets from the International Histocompatibility Workshops; and datasets from the AlleleFrequencies.net database. Altogether, these sets of data represent approximately 66,800 individuals from throughout the world. Analysis of these data revealed that the set of a rs1059449 G>A SNP, a rs41541222 G>T SNP, a reference allele G at the locus corresponding to rs1059457, a reference allele G at the locus corresponding to rs79361534, and a rs1059471 C>T SNP can be used to screen more accurately an individual carrying an HLA-A*31:01 allele.

(2) Validation of the SNPs for Screening an HLA-A*31:01 Allele

In order to screen the set of a rs1059449 G>A SNP, a rs41541222 G>T SNP, a reference allele G at the locus corresponding to rs1059457, a reference allele G at the locus corresponding to rs79361534, and a rs1059471 C>T SNP, various detection methods as well as various combination of primers and prob(s) can be designed as illustrated hereinabove.

For example, a real time PCR assay was carried out with allele specific PCR primers and probes detecting the genomic loci corresponding to SNPs rs1059449, rs41541222, rs1059457, rs79361534, and rs1059471. Exemplary PCR primers and probes used include:

Forward primer
(SEQ ID NO: 38)
5′-GATAGAGCAGGAGAGGCCT-3′
Reverse primer
(SEQ ID NO: 39)
5′-CCAGGTCCACTCGGTCAA-3′
Probe
(SEQ ID NO: 40)
5′-AGACACGGAATGTGAA-3′

As illustrated in FIG. 1, the forward primer overlaps with the locus corresponding to rs1059449 and rs41541222, and is specific for the rs1059449 G>A, and rs41541222 G>T SNP. The reverse primers overlaps with the locus corresponding to rs1059471 and is specific for the rs1059471 C>T SNP. The probe is specific for the reference allele G at the locus corresponding to rs1059457, and reference allele G at the locus corresponding to rs79361534. The probe was labeled with VIC™ on the 5′ end and TAMRA on the 3′ end. The PCR assay also included a pair of primers and a probe for amplifying beta-actin as an internal control. The sequences of the primers and probe for amplifying beta-actin are as below. The probe was labeled with FAM on the 5′ end and TAMRA on the 3′ end.

Forward primer
(SEQ ID NO: 42)
5′-GAGGCCACAGCTGCAAC-3′
Reverse primer
(SEQ ID NO: 43)
5′-GTTGGCCACGTTGTCCAT-3′
Probe
(SEQ ID NO: 44)
5′-ATACGAGTCATTCAATTC-3′
or
(SEQ ID NO: 45)
5′-GAATTGAATGACTCGTAT-3′.

Simultaneous detection of the VIC and FAM signals during the PCR assay indicates that a sample tested carried an HLA-A*31:01 allele. If only FAM was detected, but no VIC, the sample tested does not carry an HLA-A*31:01 allele. Samples were analyzed using the TaqMan Genotyper software (Version 1.3.1).

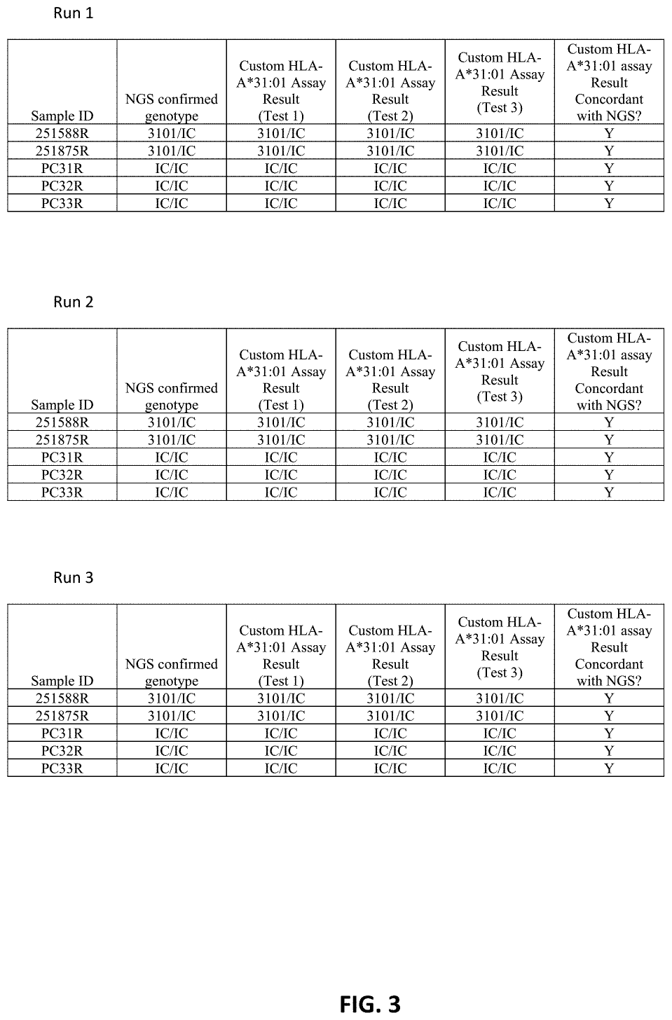

First, precision of the improved method (screening the set of a rs1059449 G>A SNP, a rs41541222 G>T SNP, a reference allele G at the locus corresponding to rs1059457, a reference allele G at the locus corresponding to rs79361534, and a rs1059471 C>T SNP) was examined. Samples collected from five individuals (PC31, PC32, PC33, 251588, & 251875 as shown in FIG. 2) were processed and the DNA was extracted as described hereinabove. Real time PCRs were run three times in the samples and the results were compared with HLA-A genotype confirmed by NGS from ProImmune. As shown in FIG. 3, the improved screening method had no variation in its ability to determine the presence or absence of HLA-A*31:01 genotype.

Next, accuracy of the improved method was examined. From the same 93 samples tested for rs1061235 as shown in Example 1, screening for a rs1059449 G>A SNP, a rs41541222 G>T SNP, a reference allele G at the locus corresponding to rs1059457, a reference allele G at the locus corresponding to rs79361534, and a rs1059471 C>T SNP was performed by real time PCR using the set primers and probes described herein. As shown in FIG. 2, the improved screening method for HLA-A*31:01 gave 100% concordance with the HLA-A genotypes confirmed by NGS.

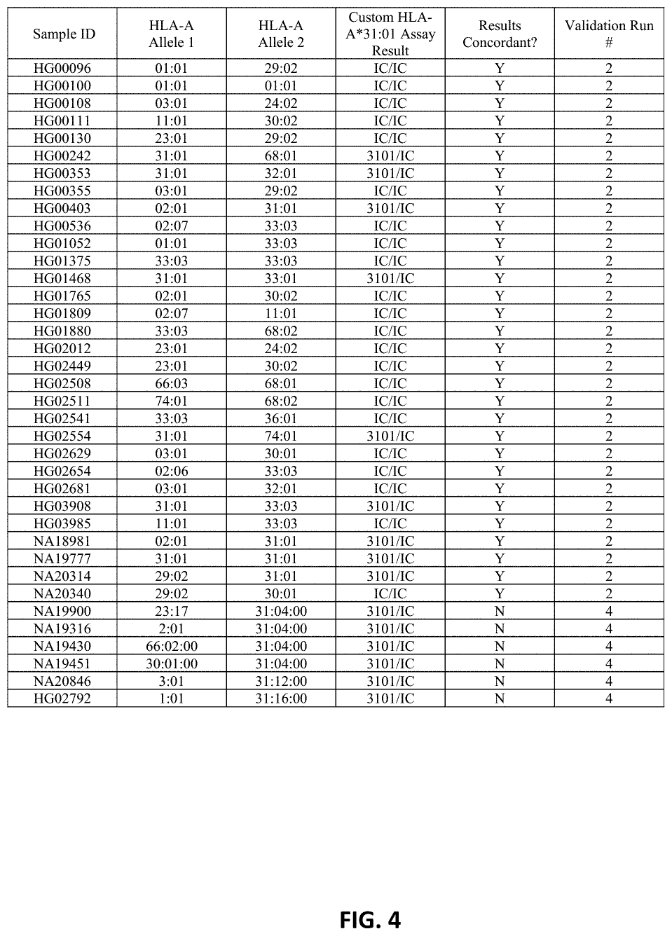

Accuracy of the improved screening method was also tested in 31 DNA samples purchased from Coriell institute with documented HLA genotypes. Among the 31 samples, 9 samples had the HLA-A*31:01 genotype, 7 samples had HLA-A*33:xx genotypes, and 15 samples had neither HLA-A*31:01 or HLA-A*33:xx genotypes. As shown in FIG. 4, the improved screening method for HLA-A*31:01 gave 100% concordance with the HLA-A genotypes in the samples purchased from Coriell institute.

(3) Theoretical False Positives Caused by Rare Non HLA-A*31:01 Genotypes

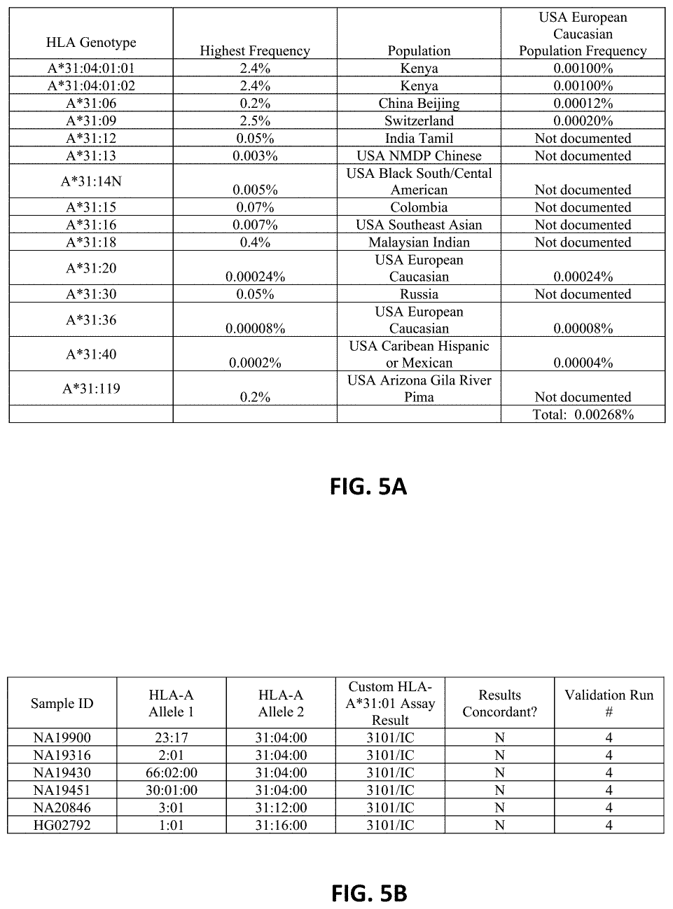

Based on theoretical sequence alignment of HLA-A genotypes, it is predicted that some rare non HLA-A*31:01 alleles would be detected by the currently used exemplary primers and probe for screening an HLA-A*31:01 allele, and could not be excluded from detection due to the sequence homology to HLA-A*31:01. As shown in FIG. 5A, these non HLA-A*31:01 genotypes are rare in the general population and depending on the ethnicity and geographical location, the percentages of these non HLA-A*31:01 genotypes varies from 2.4% to 0.00008%. Further, there is no definitive data showing that these rare non HLA-A*31:01 genotypes are associated with adverse reactions to carbamazepine. As such, the theoretical specificity for this assay is higher than 97% in populations in Kenya and Switzerland, and is higher than 99% in the other tested populations including the European Caucasian population resided in the US.

Theoretical interference from some of the non HLA-A*31:01 genotypes were confirmed. 6 samples with HLA-A*31:04, HLA-A*31:12, and HLA-A*31:16 genotypes were purchased from Coriell institute, and tested using the improved screening method for HLA-A*31:01 described herein. As shown in FIG. 5B, the 6 samples were tested positive for HLA-A*31:01 using the screening method.

(4) Limit of Detection (LoD) of the Improved Screening Method

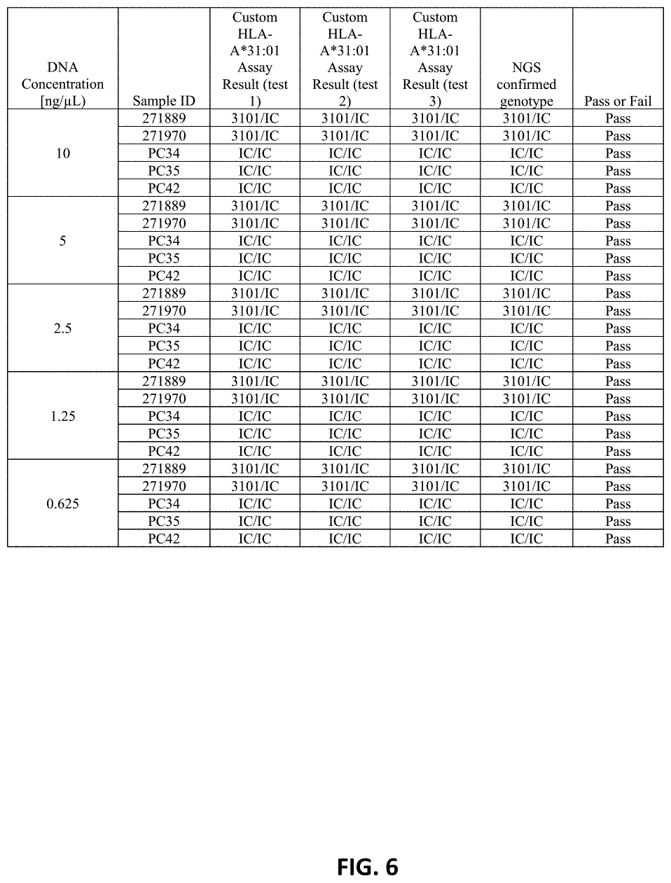

Limit of detection was determined for the improved HLA-A*31:01 screening method. DNA extracted from 5 samples (PC34, PC35, PC42, 271889, and 271970) was serially diluted to 10, 5, 2.5, 1.25, and 0.625 ng/μl, and real time PCR was performed using the primers and probe described herein. As show in FIG. 6, the screening method can be performed with 0.625 ng/μl of DNA. The limit of detection for this method is lower than 0.625 ng/μl of DNA.

1. A method of determining whether an individual carries an HLA-A*31:01 allele, comprising:
(A) performing a first polymerase chain reaction (PCR) assay designed to amplify a first genomic region from a biological sample obtained from the individual, the first genomic region encompassing the loci corresponding to SNPs rs1059449, rs41541222, rs1059457, rs79361534, and rs1059471, using a first set of PCR primers that flank the first genomic region, wherein: the first set of PCR primers comprises at least one of (i) a first primer that overlaps the locus corresponding to rs1059449 and is specific for the rs1059449 G>A SNP or (ii) a second primer that overlaps the locus corresponding to rs1059471 and is specific for the rs1059471 C>T SNP, wherein:
when the first set of PCR primers includes the first primer:
the locus corresponding to rs41541222 is either (i) overlapped by the first primer, wherein the first primer is specific for the rs41541222 G>T SNP or (ii) overlapped by a respective detection reagent in a set of one or more HLA-A*31:01 detection reagents, wherein the respective detection reagent overlaps with the locus corresponding to rs41541222 and is specific for the rs41541222 G>T SNP, and
the locus corresponding to rs1059471 is either (i) overlapped by the second allele-specific primer, which is included in the first set of primers or (ii) overlapped by a respective detection reagent in the set of one or more HLA-A*31:01 detection reagents, wherein the respective detection reagent overlaps with rs1059471 and is specific for the rs1059471 C>T SNP; and
when the first set of PCR primers includes the second primer:
the locus corresponding to rs1059449 is either (i) overlapped by the first primer, which is included in the first set of primers or (ii) overlapped by a respective detection reagent in the set of one or more HLA-A*31:01 detection reagents, wherein the respective detection reagent overlaps with rs1059449 and is specific for the rs1059449 G>A SNP, and
the locus corresponding to rs41541222 is either (i) overlapped by the first primer, wherein the first primer is specific for the rs41541222 G>T SNP or (ii) overlapped by a respective detection reagent in the set of one or more HLA-A*31:01 detection reagents, wherein the respective detection reagent overlaps with rs41541222 and is specific for the rs41541222 G>T SNP; and
(B) contacting the first PCR assay with the set of one or more HLA-A*31:01 detection reagents, wherein each respective detection reagent in the set of one or more HLA-A*31:01 detection reagents comprises a moiety that is detectable when the first genomic region is amplified and the respective detection reagent is capable of specifically binding to the first genomic region; and
(C) attempting to detect the set of one or more HLA-A*31:01 detection reagents, wherein:
a first detection reagent in the set of one or more HLA-A*31:01 detection reagents is specific for the presence of the reference G allele at the locus corresponding to the rs1059457 SNP,
a second detection reagent in the set of one or more HLA-A*31:01 detection reagents is specific for the presence of the reference G allele at the locus corresponding to the rs79361534 SNP,
when the entire set of one or more HLA-A*31:01 detection reagents is detected, the individual is deemed to be a carrier of the HLA-A*31:01 allele, and
when less than the entire set of one or more HLA-A*31:01 detection reagents is detected, the individual is deemed not to be a carrier of the HLA-A*31:01 allele, thereby determining whether the individual carries the HLA-A*31:01 allele.

2. The method of claim 1, wherein the first genomic region does not encompass the locus corresponding to the rs41562315 SNP.

3. The method of claim 1, wherein the first primer overlaps with the locus corresponding to rs41541222 and is specific for the rs41541222 G>T SNP.

4. The method of claim 1, wherein the first primer has a nucleotide sequence comprising at least 10 consecutive nucleotides of SEQ ID NO:1.

5. The method of claim 4, wherein said first primer comprises a nucleic acid sequence of SEQ ID NO:38.

6. The method of claim 1, wherein the second primer has a nucleotide sequence comprising at least 10 consecutive nucleotides of SEQ ID NO:11.

7. The method of claim 6, wherein the second primer comprises a nucleic acid sequence of SEQ ID NO:39.

8. The method of claim 1, wherein the first set of PCR primers consists of the first primer and the second primer.

9. The method of claim 1, wherein the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP.

10. The method of claim 9, wherein the single HLA-A*31:01 detection reagent comprises a nucleic acid sequence of SEQ ID NO:40 or SEQ ID NO:41.

11. The method of claim 1, wherein each respective detection reagent in the set of one or more HLA-A*31:01 detection reagents is conjugated to one or more detection label.

12. The method of claim 11, wherein the one or more detection label comprises a fluorescent dye.

13. The method of claim 12, wherein the one or more detection label comprises a matching pair of electronic energy transfer fluorophores comprising a donor fluorophore and a quencher fluorophore for the donor fluorophore.

14. The method of claim 1, wherein contacting in (B) comprises adding the set of one or more HLA-A*31:01 detection reagents to the PCR assay prior to performing (A).

15. The method of claim 1, wherein contacting in (B) comprises contacting the set of one or more HLA-A*31:01 detection reagents with a product of the PCR assay performed in (A).

16. The method of claim 1, wherein the first PCR assay is a quantitative real-time PCR assay.

17. The method of claim 1, wherein:
the first PCR assay is a multiplex PCR assay using a second set of PCR primers designed to amplify a control genomic locus from the biological sample obtained from the individual; and
the first PCR assay is contacted with a control detection reagent that binds to the control genomic locus.

18. The method of claim 17, wherein:
the set of one or more HLA-A*31:01 detection reagents consists of a single HLA-A*31:01 detection reagent that is specific for (i) the presence of the reference G allele at the locus corresponding to the rs1059457 SNP and (ii) the presence of the reference G allele at the locus corresponding to the rs79361534 SNP;
the single HLA-A*31:01 detection reagent is conjugated to a first matching pair of electronic energy transfer fluorophores comprising a first donor fluorophore and a first quencher fluorophore for the first donor fluorophore; and
the control detection reagent is conjugated to a second matching pair of electronic energy transfer fluorophores comprising a second donor fluorophore and a second quencher fluorophore for the second donor fluorophore.

19. The method of claim 18, wherein the first and the second matching pairs of electronic energy transfer fluorophores are selected from the group consisting of FAM-TAMRA, VIC-TAMRA, TET-TAMRA and JOE-TAMRA, and wherein the first and the second matching pairs of electronic energy transfer fluorophores are different.

20. The method of claim 1, further comprising determining whether the individual is a carrier of the rs1061235 A>T SNP, wherein when it is determined that the individual does not carry the rs1061235 A>T SNP, the individual is deemed not to be a carrier of the HLA-A*31:01 allele.

21-68. (canceled)
